(19) United States
(12) Patent Application Publication
(10) Pub. No.: US 2022/0251054 A1
(43) Pub. Date: Aug. 11, 2022

(54) Analogues and Methods of Treating Rett Syndrome

(71) Applicant: The Regents of the University of California, Oakland, CA (US)

(72) Inventors: William Lowry, Los Angeles, CA (US); Bennett Novitch, Los Angeles, CA (US); Michael E. Jung, Los Angeles, CA (US); Xiaoguang Liu, Los Angeles, CA (US); Ranmal Samarasinghe, Los Angeles, CA (US); Elena Korsakova, Los Angeles, CA (US)

(21) Appl. No.: 17/616,151

(22) PCT Filed: Jun. 2, 2020
(86) PCT No.: PCT/US2020/035643

§ 371 Date: Dec. 2, 2021

Related U.S. Application Data
(60) Provisional application No. 62/856,504, filed on Jun. 3, 2019.

(51) Int. Cl. C07D 277/82; A61P 25/28; C12N 5/079; G01N 33/50

(52) U.S. Cl. CPC C07D 277/82; A61P 25/28; G01N 33/5032; G01N 33/5088; C12N 5/0618

(57) ABSTRACT

Disclosed herein are Pifithrin compounds, methods of treating Rett Syndrome, brain fusion organoids comprising a fusion between a cerebral cortex (Cx) organoid and the ganglionic eminence (GE) organoid, one of which comprises, consists essentially of, or consists of neural cells having a loss of function mutation in the Methyl-CpG Binding Protein 2 (MECP2) gene, and methods of using the brain fusion organoid to screen for candidate compounds that treat, reduce, or inhibit the abnormal neural activities caused by MECP2-mutations.

### CROSS-REFERENCE TO RELATED APPLICATIONS

This application claims the benefit of U.S. Patent Application No. 62/856,504, filed Jun. 3, 2019, which is herein incorporated by reference in its entirety.

### BACKGROUND OF THE INVENTION

### 1. Field of the Invention

The field of the invention general relates to Pifithrin compounds and Rett Syndrome.

### 2. Description of the Related Art

Brain organoids represent a powerful tool for the study of neurological disease and brain development. Brain organoids are derived from embryonic stem cells (ESCs) or induced pluripotent stem cells (iPSCs) (ESCs and iPSCs are collectively referred to as “PSCs”, and human cells are referred to with a preceding “h”, e.g., hESCs, hiPSCs, and hPSCs) that self-organize into three-dimensional structures (“organoids”) with broad cellular diversity that mimics the layered organization of human brain.

### SUMMARY OF THE INVENTION

In some embodiments, the present invention relates to a compound having Formula I or Formula II.

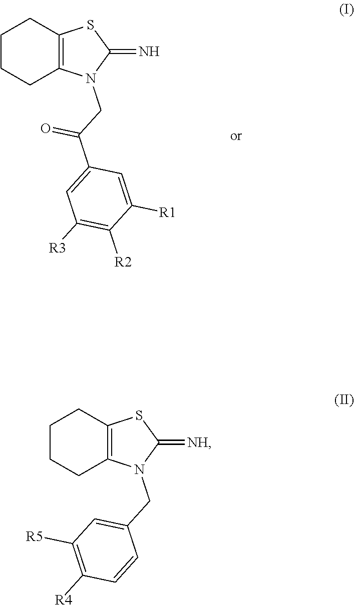

### (a) R1 is Cl or Br and R2 and R3 are H;

(b) R2 is a trimethylsilyl group (TMS), and R1 and R3 are H;

### (c) R1 and R3 are CF₃ and R2 is H;

### (d) R4 is Br and R5 is H; or

### (e) R4 and R5 are F; and

pharmaceutically acceptable salts, solvates, and prodrugs thereof.

In some embodiments, the present invention relates to compositions comprising one or more compounds a compounds having Formula I or Formula II:

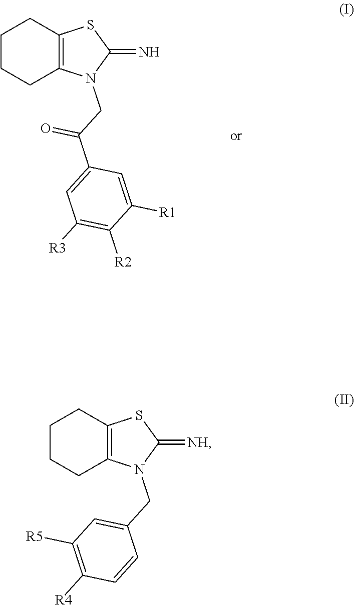

### (a) R1 is Cl or Br and R2 and R3 are H;

(b) R2 is a trimethylsilyl group (TMS), and R1 and R3 are H;

### (c) R1 and R3 are CF₃ and R2 is H;

### (d) R4 is Br and R5 is H; or

### (e) R4 and R5 are F; and

pharmaceutically acceptable salts, solvates, and prodrugs thereof, and a pharmaceutically acceptable carrier.

In some embodiments, the present invention relates to a method of treating a subject suffering from Rett Syndrome, which comprises administering to the subject at least one Pifithrin compound or a composition thereof as described herein. In some embodiments, the Pifithrin compound is 2-(2-imino-4,5,6,7-tetrahydrobenzo[d]thiazol-3(2H)-yl)-1-(p-tolyl)ethan-1-one hydrogen bromide (Pifithrin α); 2-(p-tolyl)-5,6,7,8-tetrahydroben-zo[d]imidazo[2,1-b]thiazole (Pifithrin β); N-(3-(2-oxo-2-(p-tolyl)ethyl)-4,5,6,7-tetrahydrobenzo[d]thiazol-2(3H)-ylidene)aceta-mide (Pifithrin α-Ac); 2-(2-Imino-4,5,6,7-tetrahydrobenzo[d]thiazol-3(2H)-yl)-1-(4-(trimethylsilyl)phenyl)ethan-1-one hydrogen bromide; 3-(4-bromobenzyl)-4,5,6,7-tetrahydro-benzo[d]thiazol-2(3H)-imine hydrogen bromide; 3-Benzyl-4,5,6,7-tetrahydrobenzo[d]thiazol-2(3H)-imine hydrogen bromide; 3-(4-Methylbenzyl)-4,5,6,7-tetrahydrobenzo[d]thiazol-2(3H)-imine hydrogen bromide; 3-(3,4-Difluorobenzyl)-4,5,6,7-tetrahydrobenzo[d]thiazol-2(3H)-imine hydrogen bromide; 1-(3-Fluorophenyl)-2-(2-imino-4,5,6,7-tetrahydrobenzo[d]thiazol-3(2H)-yl)ethan-1-one hydrogen bromide; 2-(2-Imino-4,5,6,7-tetrahydrobenzo[d]thiazol-3(2H)-yl)-1-(3-nitrophenyl)ethan-1-one hydrogen bromide; 2-(2-Imino-4,5,6,7-tetrahydrobenzo[d]thiazol-3(2H)-yl)-1-(4-methoxyphenyl)ethan-1-one hydrogen bromide; 1-(3-Chlorophenyl)-2-(2-imino-4,5,6,7-tetrahydrobenzo[d]thiazol-3(2H)-yl)ethan-1-one hydrogen bromide; 1-(3,4-Dichlorophenyl)-2-(2-imino-4,5,6,7-tetrahydrobenzo[d]thiazol-3(2H)-yl)ethan-1-one hydrogen bromide; 1-(3,5-Bis(trifluoromethyl)phenyl)-2-(2-imino-4,5,6,7-tetrahydrobenzo[d]thiazol-3(2H)-yl)ethan-1-onehydrogen bromide; 1-(3-Bromophenyl)-2-(2-imino-4,5,6,7-tetrahydrobenzo[d]thiazol-3(2H)-yl)ethan-1-one hydrogen bromide; or 2-(2-Imino-4,5,6,7-tetrahydrobenzo[d]thiazol-3(2H)-yl)-1-(4-(trifluoromethyl)phenyl)ethan-1-one hydrogen bromide. In some embodiments, the Pifithrin compound is 2-(2-imino-4,5,6,7-tetrahydrobenzo[d]thiazol-3(2H)-yl)-1-(p-tolyl)ethan-1-one hydrogen bromide (Pifithrin α); 2-(p-tolyl)-5,6,7,8-tetrahydroben-zo[d]imidazo[2,1-b]thiazole (Pifithrin β); N-(3-(2-oxo-2-(p-tolyl)ethyl)-4,5,6,7-tetrahydrobenzo[d]thiazol-2(3H)-ylidene)aceta-mide (Pifithrin α-Ac); or 2-(2-Imino-4,5,6,7-tetrahydrobenzo[d]thiazol-3(2H)-yl)-1-(4-(trimethylsilyl)phenyl)ethan-1-one hydrogen bromide. In some embodiments, the Pifithrin compound is 2-(2-imino-4,5,6,7-tetrahydrobenzo[d]thiazol-3(2H)-yl)-1-(p-tolyl)ethan-1-one hydrogen bromide (Pifithrin α); 3-(4-bromobenzyl)-4,5,6,7-tetrahydro-benzo[d]thiazol-2(3H)-imine hydrogen bromide; 3-Benzyl-4,5,6,7-tetrahydrobenzo[d]thiazol-2(3H)-imine hydrogen bromide; 3-(4-Methylbenzyl)-4,5,6,7-tetrahydrobenzo[d]thiazol-2(3H)-imine hydrogen bromide; 3-(3,4-Difluorobenzyl)-4,5,6,7-tetrahydrobenzo[d]thiazol-2(3H)-imine hydrogen bromide; or 1-(3-Fluorophenyl)-2-(2-imino-4,5,6,7-tetrahydrobenzo[d]thiazol-3(2H)-yl)ethan-1-one hydrogen bromide.

In some embodiments, the present invention relates to a brain fusion organoid comprising a cerebral cortex (Cx) organoid fused to a ganglionic eminence (GE) organoid. In some embodiments, the cerebral cortex (Cx) organoid and/or the ganglionic eminence (GE) organoid comprises, consists essentially of, or consists of neural cells having a loss of function mutation in the Methyl-CpG Binding Protein 2 (MECP2) gene.

In some embodiments, the present invention relates to an assay method for determining whether a given compound treats, inhibits, or reduces abnormal neural activity resulting from neural cells having a loss of function mutation in the Methyl-CpG Binding Protein 2 (MECP2) gene, which comprises contacting the brain fusion organoid as described herein with the given compound and comparing the resulting oscillatory activity with that of untreated controls.

Both the foregoing general description and the following detailed description are exemplary and explanatory only and are intended to provide further explanation of the invention as claimed. The accompanying drawings are included to provide a further understanding of the invention and are incorporated in and constitute part of this specification, illustrate several embodiments of the invention, and together with the description explain the principles of the invention.

### DESCRIPTION OF THE DRAWINGS

This invention is further understood by reference to the drawings wherein:

FIG. 1: schematically shows the generation, patterning, and fusion of dorsal cortex (Cx) and ventral ganglionic eminence (GE) organoids.

FIG. 2: Rett syndrome fusion organoids have a higher density of excitatory synapses and exhibit hypersynchronous neural network activity. Top: At about Day 100 unfused iCtrl and Mut Cx organoids show minimal expression of GAD65 expression. By contrast, about 20-25% of the cells in the Cx end of aged matched Cx+GE organoids express GAD65. n=3 organoids, 2631 cells, ns, not significant. Middle: Plots of the number of synapses per cell. Data were pooled from multiple organoids. VGLUT1/PSD95, n=3 organoids for iCtrl and n=3 for Mut, 1180 cells) VGAT/GEPHYRIN, n=4 organoids for iCtrl and n=4 organoids for Mut, 1654 cells, *P=0.0244. Mut Cx+GE organoids exhibit spontaneous synchronized Ca²⁺ transients that are not seen in iCtrl Cx+GE organoids. Bottom: Pooled data quantifications, n=7 iCtrl, n=10 Mut **P=0.0032 for synchronized transients, **P=0.0012 for multi-spiking neurons.

FIG. 3: Rett syndrome fusion organoids display GE-dependent epileptiform changes that can be partially rescued by a chemical inhibitor of p53 function. Panel a, Raw trace of a representative 10-minute LFP recording (top) and time expanded window (bottom) from unmixed Mut or iCtrl Cx+GE organoids and Mut+iCtrl mixed organoids. Panels b-c, Spectrograms and periodograms derived from the entire recordings shown in Panel a. Panel d, Morlet plot showing high frequency activity associated with the expanded time segments shown in Panel a. Panel e, Frequency histogram of interspike intervals derived from the raw trace in Panel a. Panel f, Raw trace (top), time expanded window (middle), and periodogram (bottom) from representative Mut Cx+GE organoids treated for 48-hr with vehicle (DMSO, Veh), 2 mM sodium valproate (VPA), or 10 μM Pifithrin-α (PFT-α). Panel g, Morlet plot derived from the time expanded segment in Panel f Panel h, Tabulation of the number of independent experiments that demonstrated sustained gamma oscillations (about 40-80 Hz) within the Cx+GE organoids. Chi square, iCtrl vs Mut **P=0.003, iCtrl vs iCtrl Cx+Mut GE **P=0.0012, Mut+Veh vs Mut+PFT-α, *P=0.04). Panel i, Spike frequency across multiple independent experiments ***P=0.002, *P<0.05, n=10 for Mut, n=6 for all others. Panel j, Spike Frequency following drug addition **P=0.0042, *P<0.05, n=5 for Mut+Veh, n=6 Mut+VPA, n=7 Mut+PFT-α. Color versions of these figures may be obtained from Samarasinghe, et al. (2019) bioRxiv 820183.

FIG. 4: Alternative neuronal clustering approaches result in similar cluster characteristics. Panel a, H9 hESC-derived Cx+Cx and Cx+GE organoids demonstrate similar individual neuronal activity characteristics but a non-significant (ns) trend towards increased spontaneous activity in Cx+GE organoids (n=4 Cx+Cx; n=6 Cx+GE; P=0.25). Panel b, Schematic of cluster characteristics that were derived and shown in Panels c-e. Panel c, Pooled data based on post hoc analyses utilizing neuronal Ca²⁺ activity correlations in Cx+Cx organoids versus Cx+GE organoids reveals no statistically significant changes in Ca²⁺ cluster characteristics but a trend towards smaller clusters in Cx+GE organoids (n=4 for Cx+Cx; n=6 for Cx+GE; P=0.07 for pairwise distances; P=0.56 for cluster circumference; P=0.31 for cluster area; P=0.07 for neurons per cluster). Panel d, Pooled data based on post hoc analyses utilizing neuronal Ca²⁺ activity cross correlations reveal similar clustering characteristics as when clustered using correlations in Panel c (P=0.06 for pairwise distances; P=0.52 for cluster circumference; P=0.07 for cluster area; P=0.30 for neurons per cluster).

FIG. 5: Spatially restricted microcircuit clusters in MECP2 Mut Cx+GE organoids. Pooled data for neuronal clusters derived here using Ca²⁺ activity correlations, reveal spatially restricted (smaller) microcircuit clusters with fewer average neurons per cluster in Mut compared to isoCtrl. (n=7 for iCtrl, n=10 for Mut *P<0.05).

FIG. 6: Additional independent examples of local field potential recordings. Panels a and d, Representative raw 10-minute LFP traces (top) and time expanded segments (bottom) from either unmixed iCtrl or Mut Cx+GE organoids, or Mut Cx+iCtrl GE organoids or iCtrl Cx+Mut GE organoids. Panels b and e, Morlet plots derived from the time expanded segments shown in Panels a and d. Panels c and f, Periodogram derived from the entire 10-minute traces shown in Panels a and d. Color versions of these figures may be obtained from Samarasinghe, et al. (2019) bioRxiv 820183.

FIG. 7: Tracing of network activity in WT, RETT and RETT+pifithrin organoids. PSD of a Mut Cx+GE organoids exposed to 100 μM Pifithrin-α for 48 hours shows rescue of sustained oscillatory activity. Day 100 Cx+GE organoids underwent extracellular recording to measure oscillatory activity. Untreated iCtrl Cx+GE organoids (solid line) and Mut Cx+GE organoids (RTT) exposed to 10 μM pifithrin for 48 hours (dashed line) reveal oscillatory activity at multiple frequencies. The Mut Cx+GE organoids not exposed to drug (light gray line) has no low frequency oscillatory activity.

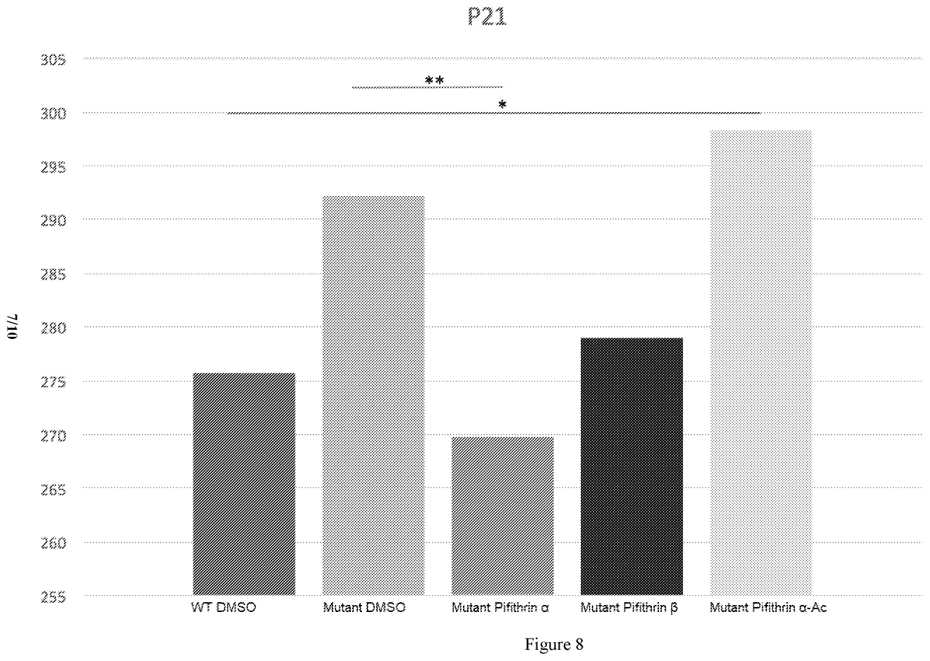

FIG. 8: Treatment with Pifithrin α and Pifithrin analogues block cell senescence due to increased p53 activity. Neurons from WT and MECP2⁻ (Mutant) cell lines were treated as indicated, and then stained for P21 protein which is a P53 target and known marker of senescence cells. WT and Mutant interneurons were treated with indicated compound (10 μM) or equivalent amount of the vehicle (DSMO) for 48 hours. Fresh media containing either DSMO or the drugs was supplied every 24 hours. Then the cells were fixed with 4% PFA for 15 minutes and permeabilized by 0.01% Triton-X-100, followed by blocking in donkey serum for 1 hour. Then p21 antibody in blocking buffer was applied for 1 hour at room temperature, followed by secondary antibody. Images were taken at 40×. Protein levels were measured by immunofluorescence. This was quantified with IMAGEJ software and the relative signal is plotted on the y-axis.

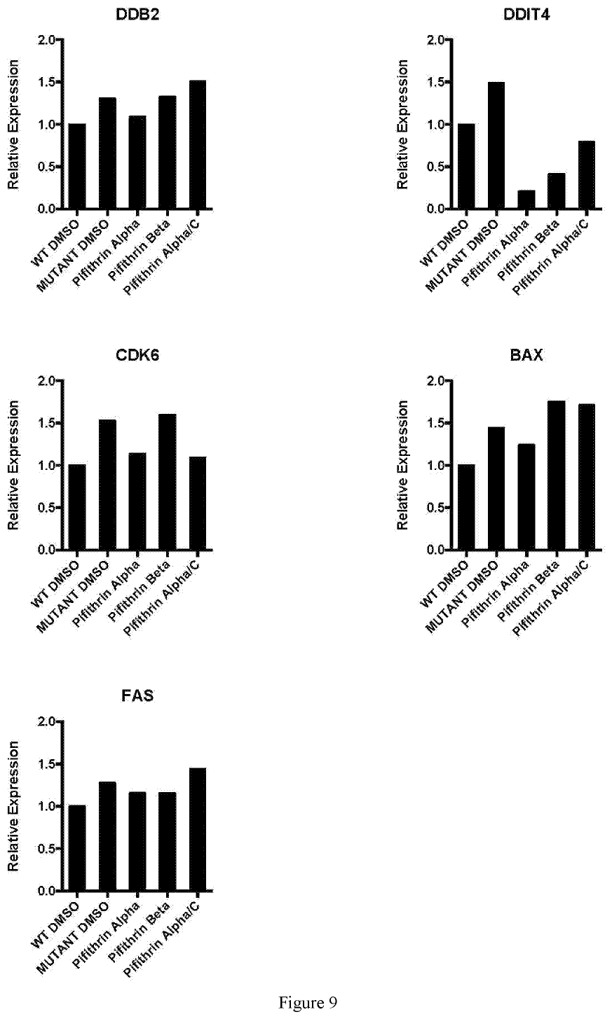

FIG. 9: Cells treated with Pifithrin α and Pifithrin analogues showed altered response to P53 at the RNA level. Neurons from WT and MECP2⁻ (Mutant) cell lines show increased levels of some P53 target genes as measured by RT-PCR. These effects were abrogated by Pifithrin α, Pifithrin β, and Pifithrin α-Ac (Pifithrin Alpha/C). Mutant IPSCs were treated with either indicated drug (10 μM) or equivalent amount of the vehicle (DSMO) for 48 hours. Fresh media containing either DMSO or the indicated Pifithrin analogue was supplied every 24 hours. Next, cells were harvested and RNA was isolated. 500 ng of RNA were used to generate cDNA, followed by RT-PCR. The relative results plotted against a housekeeping gene on the y-axis compared to control cells.

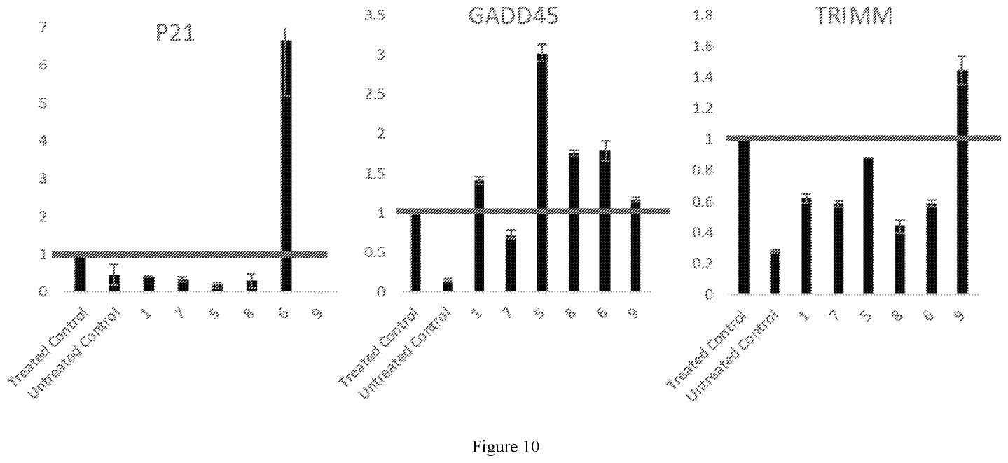

FIG. 10: Pifithrin α and Pifithrin analogues to block senescence in neurons. Cells were treated with UV irradiation to induce DNA damage and a senescence response. Cells were also pretreated with Pifithrin a (1) or a Pifithrin analogue (7=MXL007, 5=MXL005, 8=MXL008, 6=MXL006, 9=MXL009). Compounds that block senescence in response to DNA damage show decreased expression of the indicated P53 targets relative to the treated control (below line). Wildtype IPSCs were treated with either the indicated Pifithrin analogue (10 μM) or equivalent amount of the vehicle (DMSO). 24 hours later cells were washed with PBS. Next, cells in PBS were treated with UV light at 100 mJ for 60 seconds to induce DNA damage and a p53 response. Fresh media with either indicated drug (10 μM) or equivalent amount of the vehicle (DMSO) was then applied to the cells. 4 hours post UV exposure cells were harvested and RNA was isolated. 500 ng of RNA were used to generate cDNA, followed by RT-PCR for the relative expression of the indicated genes. Relative to untreated cells, one can see that these p53 target genes were induced by UV light. In addition, treatment with certain Pifithrin analogues show p53 target genes below the red bar indicating an effect on the p53 response.

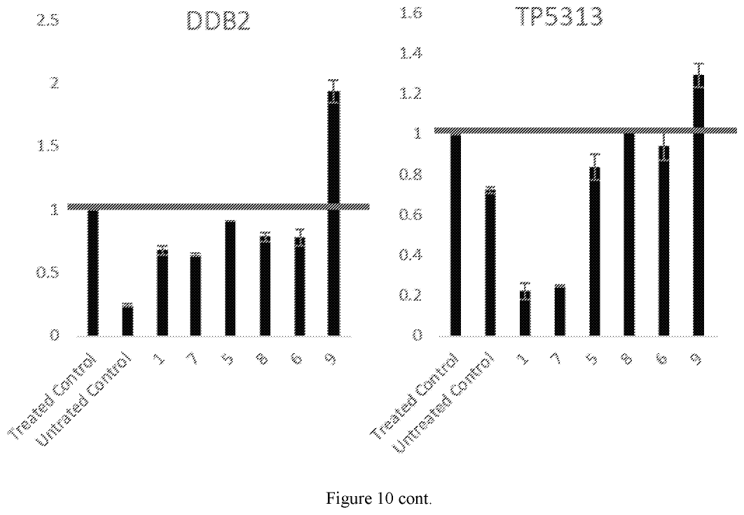

### DETAILED DESCRIPTION OF THE INVENTION

Because Rett Syndrome is associated with loss of function mutations in the Methyl-CpG Binding Protein 2 (MECP2) gene, which result in abnormal neural activity, brain fusion organoids were developed to study the abnormal neural activity associated with Rett Syndrome. Specifically, Cx+GE organoids were made from hiPSCs having MECP2 mutations. As used herein a “brain fusion organoid”, refers to a fusion of at least two different brain organoids, e.g., a fusion of a cortical organoid and a subcortical organoid. As used herein, a “brain organoid” refers spheroids and organoids resembling tissues of one or more regions of the brain. That is, a brain organoid is a three-dimensional structure comprising one or more cells that are typical of the given region of the brain. As provided herein, brain organoids are referred to by the region of the brain they resemble and brain fusion organoids are referred to by the fused organoids. For example, a cortical (or, e.g., cerebral cortex) organoid (“Cx organoid”) refers to a brain organoid that resembles the cortex of the brain, and a “Cx+GE organoid” refers to a brain fusion organoid comprising a cerebral cortex (Cx) organoid and a ganglionic eminence (GE) organoid fused together into a single organoid. Thus, Cx+GE organoids comprise excitatory neurons of the cerebral cortex and inhibitory neurons of the ganglionic eminence regions integrated together into a single organoid.

Calcium sensor imaging, unbiased post imaging algorithmic analyses, and extracellular recordings of local field potentials, indicate that “mutant” Cx+GE organoids exhibit spontaneous synchronization of calcium transients, elevated spiking, and appearance of epileptiform-like high frequency oscillations coincident with an absence of sustained low frequency and gamma oscillations as compared to “normal” Cx+GE organoids that lack MECP2 mutations. Thus, the mutant Cx+GE organoids may be used to model the abnormal neural activity associated with Rett Syndrome and screen for therapeutics that treat, reduce, or inhibit abnormal neural activity caused by MECP2 mutations.

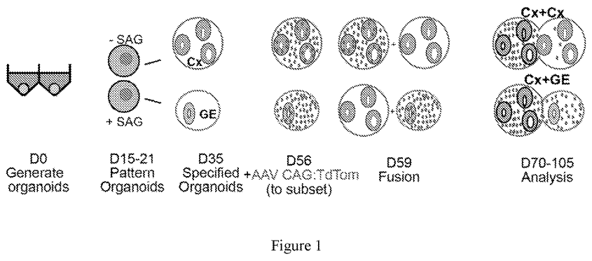

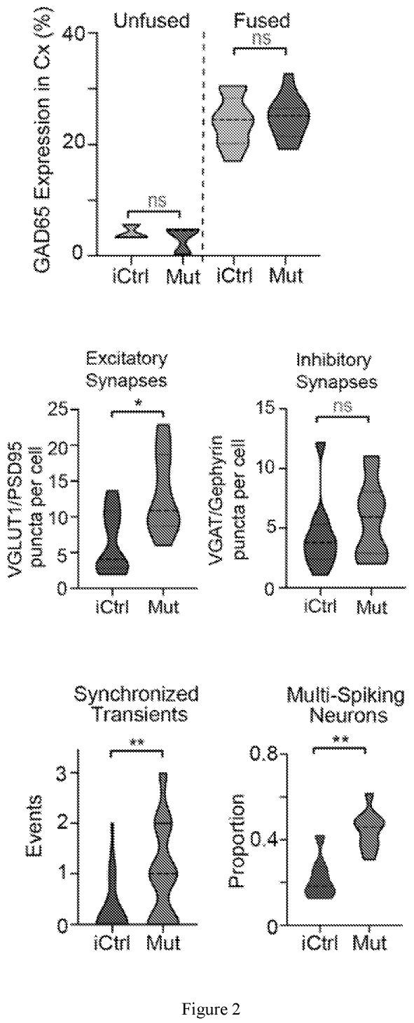

Specifically, as disclosed herein, organoid fusion techniques in the art were used to create brain fusion organoids comprising both excitatory and inhibitory neuronal subtypes as well as glia cells. Organoids were directed towards cortex (Cx) or ganglionic eminence (GE) identities through the absence or presence of Sonic hedgehog (Shh) pathway agonists (FIG. 1, Panel a). In the absence of Shh signaling, organoids predominantly exhibited cortical character including expression of the apical and basal radial glial progenitor marker PAX6, the intermediate progenitor marker TBR2 (EOMES), deep cortical plate markers including TBR1, CTIP2 (BCL11B), and BHLHB5 (BHLHE22), and superficial layer markers such as SATB2, and BRN2 (POU3F2) (FIG. 1, Panel b; FIG. 2, Panel a). Shh pathway-stimulated organoids, by contrast, expressed canonical GE progenitor and migratory interneuron markers such as NKX2.1, DLX1, DLX2, and OLIG2. Over time in culture, many neurons within the GE organoids robustly expressed the GABAergic neuron marker GAD65 along with a variety of interneuron subtype markers including somatostatin (SST), calretinin, and calbindin.

In the developing forebrain in vivo, GE-derived interneurons migrate tangentially into the adjacent cortex and functionally integrate into cortical neural networks, a process that can be recapitulated in vitro. Using adeno-associated virus (AAV)-TdTomato labeling of the GE organoid before fusion of the Cx and GE organoids (“Cx+GE fusion”), widespread migration of cells originating from the GE and dispersion within the adjacent Cx two weeks after fusion was observed (FIG. 1, Panels a,c). Minimal TdTomato⁺ cell migration was seen in controls (Cx+Cx organoids or Cx+GE organoids, where Cx was pre-labeled with AAV-TdTomato) (FIG. 1, Panel c). Immunohistochemical analyses of the cortical aspect of Cx+GE organoids revealed the intermingling of Cx-derived excitatory neurons, exemplified by SATB2 which is not expressed within GE organoids, and inhibitory interneurons identified by GAD65 and DLX5 co-staining (FIG. 1, Panel d). By contrast, Cx+Cx organoids only expressed the neuronal marker SATB2 with few if any GAD65⁺ DLX5⁺ cells (FIG. 1, Panel d). The integration of excitatory and inhibitory interneurons within the Cx+GE organoids was further confirmed by the prominence of both excitatory synapses, distinguished by colocalization of the glutamatergic presynaptic protein vesicular glutamate receptors-1 or 2 (VGLUT1 or VGLUT2) and their post synaptic partner, post synaptic density-95 (PSD95), and inhibitory synapses, visualized by vesicular gamma-aminobutyric acid (GABA) transporter (VGAT) and gephryin staining. Cx+Cx organoids, in comparison, predominantly contained only excitatory synapses.

To determine the range of physiological activity in the brain fusion organoids, live two-photon based calcium imaging of intact organoids and extracellular recordings of local field potentials (LFPs) were used. Constrained non-negative matrix factorization extended (CNMF-E) methods for calcium signal processing were applied, which permitted unbiased categorization of single cell calcium dynamics into functional microcircuit clusters. In combination with LFP data, this approach allows the characterization of brain organoid physiological activity at the single cell, microcircuit, and network levels. After infection with AAV-GCaMP6f virus, spontaneous calcium activity was measured as changes in GCaMP6f fluorescence (ΔF/F). Both Cx+Cx organoids and Cx+GE organoids showed comparable spontaneous neural activities. To determine functional GABAergic interneuron-glutamatergic cell connectivity, either the GABA_{A} receptor antagonist bicuculline methiodide (BMI) or Gabazine was added and epochs of nearly complete synchronization of calcium transients was observed in Cx+GE organoids, with no such effect in Cx+Cx organoids. Hierarchical clustering revealed large groups of neurons with highly correlated activity in Cx+GE organoids following BMI treatment, while only small groups were observed in the Cx+Cx organoids.

LFPs in the fused organoids were measured and simultaneous, sustained oscillations were found at multiple frequencies from 1-100 Hz in Cx+GE organoids, yet no discernible oscillatory activity in Cx+Cx organoids was observed. Together, these data show that interneurons uniquely entrain the behavior of excitatory cells in Cx+GE organoids and that Cx+GE organoids are capable of producing complex oscillations akin to those observed by extra- and intracranial recordings of the intact human brain.

### Rett Syndrome Neural Activity Modeling

Rett Syndrome is an X-linked neurodevelopmental disorder in which affected females exhibit motor delays, cognitive and neuropsychiatric disturbances, autism, and epilepsy. Rett is typically caused by a mutation in the MECP2 gene on the X chromosome, and affected females exhibit symptoms as early as seven months of age. While neuroanatomical changes in dendritic arborization and spine density have been reported in multiple models, gross anatomical changes such as microcephaly are less prevalent. Therefore, hiPSC derived Cx and GE organoids comprising MECP2 mutations were constructed to determine whether they can be used to model the pathophysiological abnormalities associated with Rett Syndrome.

Indeed, the mutant hiPSC derived Cx and GE organoids (“Mut” or “Mut Cx+GE organoids”, which comprise a Cx organoid having one or more mutations (“Mut Cx organoid”) fused to a GE organoid having one or more mutations (“Mut CE organoid”) exhibited similar cytoarchitecture and cell composition as isogenically matched non-mutant hiPSC derived organoids (“iCtrl” or “iCtrl Cx+GE organoids”) (FIG. 2, Panels a,b). The cortex region of both iCtrl and Mut Cx+GE organoids also contained similar percentages of GAD65⁺ interneurons (mean about 25%; FIG. 2, Panels c,d), comparable to the percentages reported in most mammalian species. Staining for the excitatory synaptic proteins VGLUT1 and PSD95 was slightly increased in Mut Cx+GE organoids relative to iCtrl, while inhibitory synaptic markers (VGAT/gephyrin) was not significantly different (FIG. 2, Panels e,f).

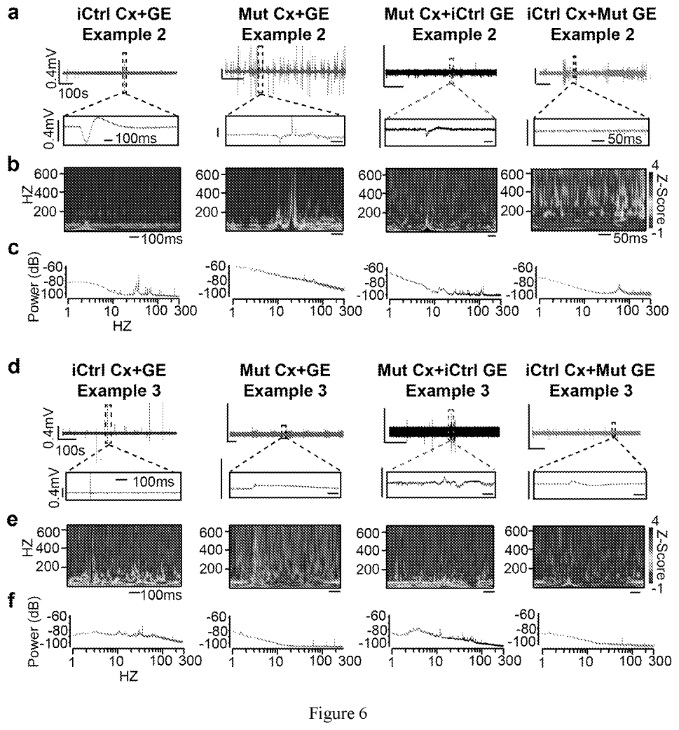

More striking, however, were activity differences revealed though GCaMP6f imaging. Mut Cx+GE organoids exhibited epochs of spontaneously synchronized calcium transients (FIG. 2, Panel g) reminiscent of the synchronizations observed following administration of GABA_{A} receptor antagonists to control samples and the epileptiform changes seen in murine models of Rett Syndrome. Increased synchronization of calcium transients in Mut Cx+GE organoids accompanied by reductions was observed in both the localization of microcircuit clusters and the number of neurons within each cluster (FIG. 5).

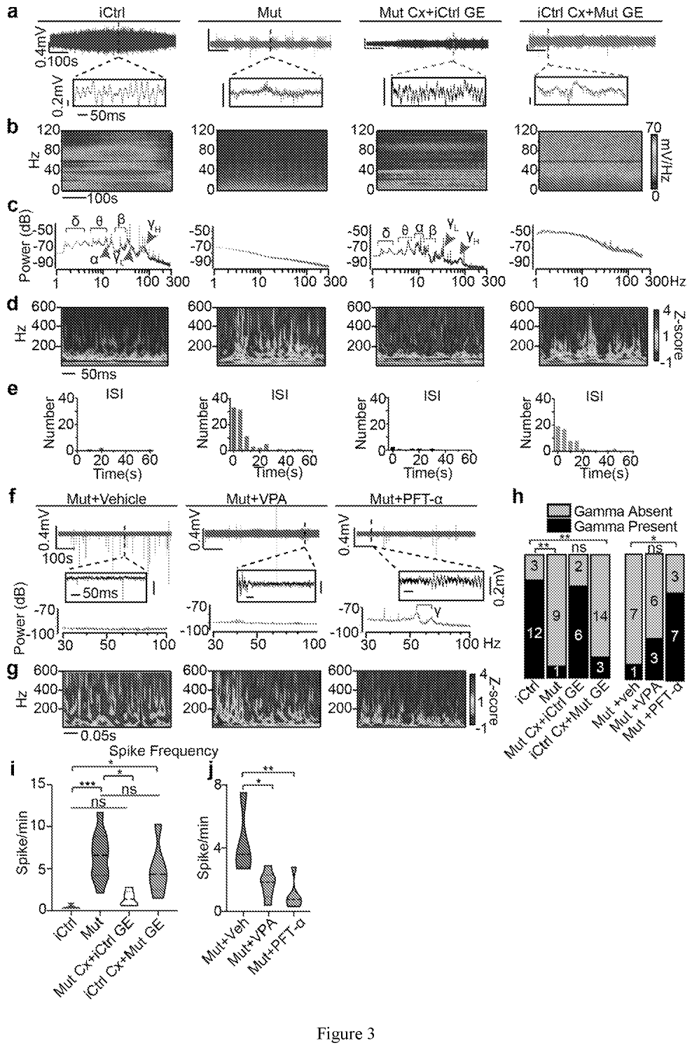

LFP recordings of iCtrl Cx+GE organoids demonstrated infrequent spikes and sustained low frequency and gamma oscillations with few incidences of higher frequency oscillations (>about 100 Hz, FIG. 3, Panels a-e). By contrast, Mut Cx+GE organoids lacked low frequency and gamma oscillations, and instead exhibited recurring epileptiform-appearing spikes and high frequency oscillations (HFOs, about 200-500 Hz; FIG. 3, Panels a-e; FIG. 5). Hypersynchrony, HFOs, and spikes seen in Mut Cx+GE organoids are all consistent with electrographic changes observed in human epilepsy. Indeed, electroencephalographic abnormalities were documented in the Rett patient whose hiPSCs were used in this study.

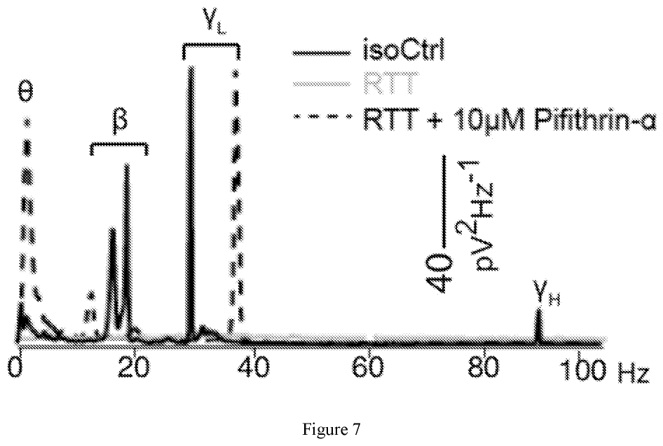

As the Cx and GE organoids are respectively enriched in excitatory versus inhibitory interneurons, control organoids (“iCtrl Cx+GE organoids”) and mixed mutant brain organoids (“Mut+iCtrl mixed organoids”, which comprise an organoid that lacks any mutations fused to another organoid that has one or more mutations), were generated. Exemplary Mut+iCtrl mixed organoids include “Mut Cx+iCtrl GE organoids”, which are brain fusion organoids comprising a Cx organoid having one or more mutations (“Mut Cx organoid”) fused to a GE organoid lacking mutations (“iCtrl GE organoid”), and “iCtrl Cx+Mut GE organoids” which comprise a Cx organoid lacking mutations (“iCtrl Cx organoid”) fused to a GE organoid having one or more mutations (“Mut GE organoid”). Mut Cx+iCtrl GE organoids displayed an LFP profile nearly identical to unmixed iCtrl Cx+GE organoids (FIG. 3, Panels a-e; FIG. 6). In contrast, iCtrl Cx+Mut GE organoids demonstrated frequent spikes and HFOs along with deficits in distinct lower frequency activity, similar to unmixed Mut Cx+GE organoids (FIG. 3, Panels a-e; FIG. 6).

Pooled data from multiple independent experiments further supported the role of Mut GE-derived interneurons driving phenotypic neural abnormalities of Rett Syndrome. The overall spike frequency was significantly increased in unmixed Mut Cx+GE organoids and iCtrl Cx+Mut GE organoids compared to iCtrl Cx+GE organoids or Mut Cx+iCtrl GE organoids (FIG. 3, Panel i). Both human and murine studies have found an inverse relationship between gamma band power and epileptiform discharges. Gamma oscillations are thought to require complex inhibitory-excitatory network interactions that are highly prone to disruption by epileptic discharges. Gamma oscillations in unmixed iCtrl Cx+GE organoids and Mut Cx+iCtrl GE organoids and significant reductions in gamma waves in Mut Cx+GE organoids and iCtrl Cx+Mut GE organoids were observed (FIG. 3, Panel h).

To determine whether brain fusion organoids can be used to screen for candidate compounds and therapies that will likely treat Rett Syndrome, Mut Cx+GE organoids were treated with a broad-spectrum anti-seizure medications (ASMs), sodium valproate (VPA), and the putative p53 inhibitor pifithrin-α, to determine what effect, if any, they had on abnormal oscillations. Exposure to VPA significantly reduced spiking activity in the Mut Cx+GE organoids (FIG. 3, Panels f,i), though it neither decreased HFOs nor restored lower frequency oscillations (FIG. 3, Panels g,h). Pifithrin-α similarly reduced spike frequency, but remarkably also suppressed HFOs and resulted in the re-emergence of gamma oscillations (FIG. 3, Panels f-i). Therefore, Pifithrin analogues were made and their impact on the abnormal neural activities caused by MECP2 mutations was similarly evaluated.

### Compositions

Compositions, including pharmaceutical compositions, comprising one or more Pifithrin compounds are contemplated herein. The term “pharmaceutical composition” refers to a composition suitable for pharmaceutical use in a subject. A composition generally comprises an effective amount of an active agent and a diluent and/or carrier. A pharmaceutical composition generally comprises a therapeutically effective amount of an active agent and a pharmaceutically acceptable carrier.

As used herein, an “effective amount” refers to a dosage or amount sufficient to produce a desired result. The desired result may comprise an objective or subjective change as compared to a control in, for example, in vitro assays, and other laboratory experiments. As used herein, a “therapeutically effective amount” refers to an amount that may be used to treat, prevent, or inhibit a given disease or condition in a subject as compared to a control, such as a placebo. Again, the skilled artisan will appreciate that certain factors may influence the amount required to effectively treat a subject, including the degree of the condition or symptom to be treated, previous treatments, the general health and age of the subject, and the like. Nevertheless, effective amounts and therapeutically effective amounts may be readily determined by methods in the art.

The one or more Pifithrin compounds may be administered, preferably in the form of pharmaceutical compositions, to a subject. Preferably the subject is mammalian, more preferably, the subject is human. Preferred pharmaceutical compositions are those comprising at least one Pifithrin compound in a therapeutically effective amount and a pharmaceutically acceptable vehicle. In some embodiments, a therapeutically effective amount of a Pifithrin compound ranges from about 0.01 to about 10 mg/kg body weight, about 0.01 to about 3 mg/kg body weight, about 0.01 to about 2 mg/kg, about 0.01 to about 1 mg/kg, or about 0.01 to about 0.5 mg/kg body weight for parenteral formulations. Therapeutically effective amounts for oral administration may be up to about 10-fold higher. It should be noted that treatment of a subject with a therapeutically effective amount may be administered as a single dose or as a series of several doses. The dosages used for treatment may increase or decrease over the course of a given treatment. Optimal dosages for a given set of conditions may be ascertained by those skilled in the art using dosage-determination tests and/or diagnostic assays in the art. Dosage-determination tests and/or diagnostic assays may be used to monitor and adjust dosages during the course of treatment.

Pharmaceutical compositions may be formulated for the intended route of delivery, including intravenous, intramuscular, intra peritoneal, subcutaneous, intraocular, intrathecal, intraarticular, intrasynovial, cisternal, intrahepatic, intralesional injection, intracranial injection, infusion, and/or inhaled routes of administration using methods known in the art. Pharmaceutical compositions may include one or more of the following: pH buffered solutions, adjuvants (e.g., preservatives, wetting agents, emulsifying agents, and dispersing agents), liposomal formulations, nanoparticles, dispersions, suspensions, or emulsions, as well as sterile powders for reconstitution into sterile injectable solutions or dispersions. The compositions and formulations may be optimized for increased stability and efficacy using methods in the art. See, e.g., Carra et al., (2007) Vaccine 25:4149-4158.

The compositions may be administered to a subject by any suitable route including oral, transdermal, subcutaneous, intranasal, inhalation, intramuscular, and intravascular administration. It will be appreciated that the preferred route of administration and pharmaceutical formulation will vary with the condition and age of the subject, the nature of the condition to be treated, the therapeutic effect desired, and the particular Pifithrin compound used.

As used herein, a “pharmaceutically acceptable vehicle” or “pharmaceutically acceptable carrier” are used interchangeably and refer to solvents, dispersion media, coatings, antibacterial and antifungal agents, isotonic and absorption delaying agents, and the like, that are compatible with pharmaceutical administration and comply with the applicable standards and regulations, e.g., the pharmacopeial standards set forth in the United States Pharmacopeia and the National Formulary (USP-NF) book, for pharmaceutical administration. Thus, for example, unsterile water is excluded as a pharmaceutically acceptable carrier for, at least, intravenous administration. Pharmaceutically acceptable vehicles include those known in the art. See, e.g., Remington: The Science and Practice of Pharmacy 20th ed (2000) Lippincott Williams & Wilkins, Baltimore, Md.

A “pharmaceutically acceptable solvate” refers to a solvate form of a specified compound that retains the biological effectiveness of such compound. Examples of solvates include compounds of the invention in combination with water, isopropanol, ethanol, methanol, dimethyl sulfoxide, ethyl acetate, acetic acid, ethanolamine, or acetone. Those skilled in the art of organic chemistry will appreciate that many organic compounds can form complexes with solvents in which they are reacted or from which they are precipitated or crystallized. These complexes are known as “solvates”. For example, a complex with water is known as a “hydrate”. Solvates of compounds of formulas I and II are within the scope of the invention. It will also be appreciated by those skilled in organic chemistry that many organic compounds can exist in more than one crystalline form. For example, crystalline form may vary from solvate to solvate. Thus, all crystalline forms of the compounds of formulas I and II or the pharmaceutically acceptable solvates thereof are contemplated herein.

The term “pharmaceutically acceptable salts” refers to salt forms that are pharmacologically acceptable and substantially non-toxic to the subject being treated with the compound of the invention. Pharmaceutically acceptable salts include conventional acid-addition salts or base-addition salts formed from suitable non-toxic organic or inorganic acids or inorganic bases. Exemplary acid-addition salts include those derived from inorganic acids such as hydrochloric acid, hydrobromic acid, hydroiodic acid, sulfuric acid, sulfamic acid, phosphoric acid, and nitric acid, and those derived from organic acids such as p-toluenesulfonic acid, methanesulfonic acid, ethane-disulfonic acid, isethionic acid, oxalic acid, p-bromophenylsulfonic acid, carbonic acid, succinic acid, citric acid, benzoic acid, 2-acetoxybenzoic acid, acetic acid, phenylacetic acid, propionic acid, glycolic acid, stearic acid, lactic acid, malic acid, tartaric acid, ascorbic acid, maleic acid, hydroxymaleic acid, glutamic acid, salicylic acid, sulfanilic acid, and fumaric acid. Exemplary base-addition salts include those derived from ammonium hydroxides (e.g., a quaternary ammonium hydroxide such as tetramethylammonium hydroxide), those derived from inorganic bases such as alkali or alkaline earth-metal (e.g., sodium, potassium, lithium, calcium, or magnesium) hydroxides, and those derived from non-toxic organic bases such as basic amino acids.

“A pharmaceutically acceptable prodrug” is a compound that may be converted under physiological conditions or by solvolysis to the specified compound or to a pharmaceutically acceptable salt of such compound. “A pharmaceutically active metabolite” refers to a pharmacologically active product produced through metabolism in the body of a specified compound or salt thereof. Prodrugs and active metabolites of a compound may be identified using routine techniques known in the art. See, e.g., Bertolini, G. et al., (1997) J. Med. Chem. 40:2011-2016; Shan, D. et al., J. Pharm. Sci., 86(7):765-767; Bagshawe K., (1995) Drug Dev. Res. 34:220-230; Bodor, N., (1984) Advances in Drug Res. 13:224-331; Bundgaard, H., Design of Prodrugs (Elsevier Press, 1985) and Larsen, I. K., Design and Application of Prodrugs, Drug Design and Development (Krogsgaard-Larsen et al., eds., Harwood Academic Publishers, 1991).

The pharmaceutical compositions may be provided in dosage unit forms. As used herein, a “dosage unit form” refers to physically discrete units suited as unitary dosages for the subject to be treated; each unit containing a predetermined quantity of the one or more Pifithrin compound calculated to produce the desired therapeutic effect in association with the required pharmaceutically acceptable carrier. The specification for the dosage unit forms of the invention are dictated by and directly dependent on the unique characteristics of the given Pifithrin compound and desired therapeutic effect to be achieved, and the limitations inherent in the art of compounding such an active compound for the treatment of individuals.

Toxicity and therapeutic efficacy of Pifithrin compounds according to the instant invention and compositions thereof can be determined using cell cultures and/or experimental animals and pharmaceutical procedures in the art. For example, one may determine the lethal dose, LC₅₀ (the dose expressed as concentration x exposure time that is lethal to 50% of the population) or the LD₅₀ (the dose lethal to 50% of the population), and the ED₅₀ (the dose therapeutically effective in 50% of the population) by methods in the art. The dose ratio between toxic and therapeutic effects is the therapeutic index and it can be expressed as the ratio LD₅₀/ED₅₀. Pifithrin compounds which exhibit large therapeutic indices are preferred. While Pifithrin compounds that result in toxic side-effects may be used, care should be taken to design a delivery system that targets such compounds to the site of treatment to minimize potential damage to uninfected cells and, thereby, reduce side-effects.

The data obtained from the cell culture assays and animal studies can be used in formulating a range of dosages for use in humans. Preferred dosages provide a range of circulating concentrations that include the ED₅₀ with little or no toxicity. The dosage may vary depending upon the dosage form employed and the route of administration utilized. Therapeutically effective amounts and dosages of one or more Pifithrin compounds can be estimated initially from cell culture assays. A dose may be formulated in animal models to achieve a circulating plasma concentration range that includes the IC₅₀ (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography. Additionally, a dosage suitable for a given subject can be determined by an attending physician or qualified medical practitioner, based on various clinical factors.

The following examples are intended to illustrate but not to limit the invention.

### Examples

### Pifithrin and Pifithrin Analogues

Pifithrin α is an effective P53 inhibitor, but has a short half-life (degrades to Pifithrin β), and does not cross the blood-brain-barrier (BBB). Pifithrin β is more stable, but not predicted to cross BBB. Therefore, a variety of Pifithrin analogues were designed and tested. Pifithrin α-Ac (MXL003) was designed to be a pro-drug to release Pifithrin α once released into the brain. Pifithrin-TMS (MXL004) adds a silicon group to increase the lipophilicity to help it cross the BBB.

### A. Synthesis of 2-(2-imino-4,5,6,7-tetrahydrobenzo[d]thiazol-3(2H)-yl)-1-(p-tolyl)ethan-1-one Hydrogen Bromide (Pifithrin α, MXL001)

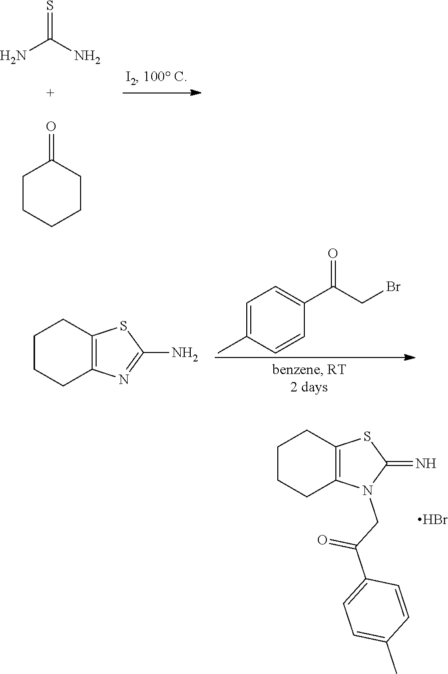

A sealed tube containing cyclohexanone (10 mmol, 1.04 mL), thiourea (20 mmol, 1.52 g) and iodine (10 mmol, 2.54 g) was heated at 100° C. for 6 h. After the reaction cooled, 20 mL hot water was added to dissolve the reaction mixture. Sodium bicarbonate powder was added to neutralize the solution. The resulting solution was extracted by diethyl ether (30 mL×3). The combined organic phase was dried over Na₂SO₄ and concentrated in vacuo. The residue was purified by column chromatography (2:1 hexanes:ethyl acetate), which generated 720 mg (47% yield) of the desired product, 4,5,6,7-tetrahydrobenzo[d]thiazol-2-amine.

A mixture of 4,5,6,7-tetrahydrobenzo[d]thiazol-2-amine (1 mmol, 154 mg) and 2-bromo-1-(p-tolyl)ethan-1-one (1 mmol, 213 mg) in dry benzene (4 mL) was stirred at 21° C. for 2 days. The white precipitate was filtered and washed with benzene (2 mL×2), which generated 256 mg (70% yield) of the desired product, 2-(2-imino-4,5,6,7-tetrahydrobenzo[d]thiazol-3(2H)-yl)-1-(p-tolyl)ethan-1-one hydrogen bromide (Pifithrin α, MXL001) as a white solid.

¹H NMR (500 MHz, DMSO-d₆) δ 9.50 (br s, 2H), 7.93 (d, J=7.9 Hz, 2H), 7.42 (d, J=7.9 Hz, 2H), 5.70 (s, 2H), 2.53 (m, 2H), 2.40 (s, 3H), 2.30 (m, 2H), 1.71 (m, 4H).

¹³C NMR (126 MHz, DMSO-d₆) δ 190.6, 168.3, 145.7, 135.1, 131.7, 129.9, 129.1, 115.0, 52.4, 22.8, 22.6, 22.4, 21.8, 21.3.

### B. The Following Analogues (MXL004, MXL009, MXL010, MXL011, MXL012, MXL013, MXL014, MXL015 and MXL016) were Prepared Using a Similar Synthetic Route as for Pifithrin α

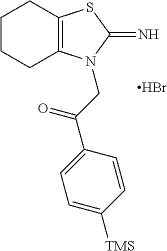 [SMILES: C[Si](C)(C)c1ccc(C(=O)Cn2c3c(sc2=N)CCCC3)cc1 ; Br]

### 2-(2-Imino-4,5,6,7-tetrahydrobenzo[d]thiazol-3(2H)-yl)-1-(4-(trimethylsilyl)phenyl)ethan-1-one Hydrogen Bromide (MXL004)

¹H NMR (500 MHz, DMSO-d₆) δ 9.55 (br s, 2H), 8.00 (d, J=8.1 Hz, 2H), 7.75 (d, J=8.1 Hz, 2H), 5.77 (s, 2H), 2.53 (m, 2H), 2.31 (m, 2H), 1.70 (m, 4H), 0.27 (s, 9H).

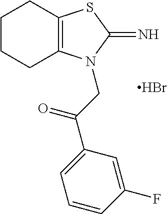 [SMILES: N=c1sc2c(n1CC(=O)c1cccc(F)c1)CCCC2 ; Br]

### 1-(3-Fluorophenyl)-2-(2-imino-4,5,6,7-tetrahydrobenzo[d]thiazol-3(2H)-yl)ethan-1-one Hydrogen Bromide (MXL009)

¹H NMR (500 MHz, DMSO-d₆) δ 9.54 (br s, 2H), 7.89 (d, J=7.7 Hz, 1H), 7.85 (dd, J=9.3, 2.0 Hz, 1H), 7.68 (m, 1H), 7.62 (m, 1H), 5.76 (s, 2H), 2.48 (m, 2H), 2.33 (m, 2H), 1.71 (m, 4H).

¹³C NMR (126 MHz, DMSO-d₆) δ 190.0, 168.4, 162.6 (d, J_{C-F}=245.5 Hz), 136.3 (d, J_{C-F}=6.6 Hz), 135.0, 131.7 (d, J_{C-F}=7.8 Hz), 125.2 (d, J_{C-F}=2.6 Hz), 121.8 (d, J_{C-F}=21.3 Hz), 115.6 (d, J_{C-F}=22.7 Hz), 115.1, 52.8, 22.8, 22.7, 22.4, 21.3.

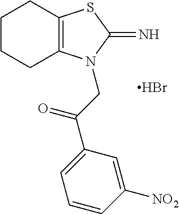 [SMILES: N=c1sc2c(n1CC(=O)c1cccc([N+](=O)[O-])c1)CCCC2 ; Br]

### 2-(2-Imino-4,5,6,7-tetrahydrobenzo[d]thiazol-3(2H)-yl)-1-(3-nitrophenyl)ethan-1-one Hydrogen Bromide (MXL010)

¹H NMR (500 MHz, DMSO-d₆) δ 9.52 (br s, 2H), 8.75 (m, 1H), 8.57 (m, 1H), 8.43 (m, 1H), 7.92 (m, 1H), 5.84 (s, 2H), 2.54 (m, 2H), 2.35 (m, 2H), 1.71 (m, 4H).

¹³C NMR (126 MHz, DMSO-d₆) δ 190.0, 168.4, 148.4, 135.5, 135.1, 131.2, 129.0, 128.8, 123.4, 115.0, 52.8, 22.8, 22.7, 22.4, 21.3.

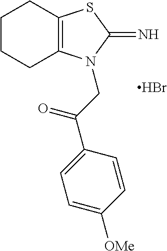 [SMILES: COc1ccc(C(=O)Cn2c3c(sc2=N)CCCC3)cc1 ; Br]

### 2-(2-Imino-4,5,6,7-tetrahydrobenzo[d]thiazol-3(2H)-yl)-1-(4-methoxyphenyl)ethan-1-one Hydrogen Bromide (MXL011)

¹H NMR (500 MHz, DMSO-d₆) δ 9.47 (br s, 2H), 8.00 (d, J=8.9 Hz, 2H), 7.13 (d, J=8.9 Hz, 2H), 5.67 (s, 2H), 3.86 (s, 3H), 2.53 (m, 2H), 2.29 (m, 2H), 1.71 (m, 4H).

¹³C NMR (126 MHz, DMSO-d₆) δ 189.4, 168.3, 164.6, 135.1, 131.4, 127.6, 114.9, 114.7, 56.3, 52.1, 22.8, 22.7, 22.4, 21.3.

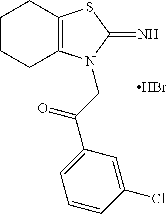 [SMILES: N=c1sc2c(n1CC(=O)c1cccc(Cl)c1)CCCC2 ; Br]

### 1-(3-Chlorophenyl)-2-(2-imino-4,5,6,7-tetrahydrobenzo[d]thiazol-3(2H)-yl)ethan-1-one Hydrogen Bromide (MXL012)

¹H NMR (500 MHz, DMSO-d₆) δ 9.53 (br s, 2H), 8.07 (s, 1H), 7.98 (m, 1H), 7.82 (ddd, J=8.0, 2.1, 0.9 Hz, 1H), 7.65 (app. t, J=8.0 Hz, 1H), 5.76 (s, 2H), 2.53 (m, 2H), 2.33 (m, 2H), 1.70 (m, 4H).

¹³C NMR (126 MHz, DMSO-d₆) δ 190.4, 168.3, 136.1, 135.1, 134.5, 134.2, 131.4, 128.8, 127.5, 115.0, 52.7, 22.8, 22.7, 22.4, 21.3.

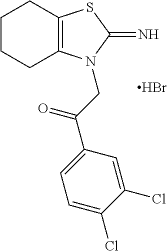 [SMILES: N=c1sc2c(n1CC(=O)c1ccc(Cl)c(Cl)c1)CCCC2 ; Br]

### 1-(3,4-Dichlorophenyl)-2-(2-imino-4,5,6,7-tetrahydrobenzo[d]thiazol-3(2H)-yl)ethan-1-one Hydrogen Bromide (MXL013)

¹H NMR (500 MHz, DMSO-d₆) δ 9.52 (br s, 2H), 8.27 (d, J=1.8 Hz, 1H), 7.97 (dd, J=8.4, 1.8 Hz, 1H), 7.92 (d, 1H, J=8.4 Hz, 1H), 5.76 (s, 2H), 2.53 (m, 2H), 2.33 (m, 2H), 1.71 (m, 4H).

¹³C NMR (126 MHz, DMSO-d₆) δ 189.8, 168.4, 137.6, 135.0, 134.5, 132.3, 131.7, 131.0, 128.9, 115.0, 52.7, 22.8, 22.7, 22.4, 21.3.

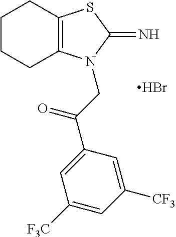 [SMILES: N=c1sc2c(n1CC(=O)c1cc(C(F)(F)F)cc(C(F)(F)F)c1)CCCC2 ; Br]

### 1-(3,5-Bis(trifluoromethyl)phenyl)-2-(2-imino-4,5,6,7-tetrahydrobenzo[d]thiazol-3(2H)-yl)ethan-1-onehydrogen Bromide (MXL014)

¹H NMR (500 MHz, DMSO-d₆) δ 9.53 (br s, 2H), 8.59 (s, 2H), 8.55 (s, 1H), 5.90 (s, 2H), 2.54 (m, 2H), 2.38 (m, 2H), 1.70 (m, 4H).

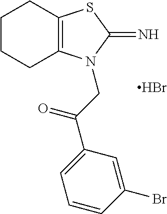 [SMILES: N=c1sc2c(n1CC(=O)c1cccc(Br)c1)CCCC2 ; Br]

### 1-(3-Bromophenyl)-2-(2-imino-4,5,6,7-tetrahydrobenzo[d]thiazol-3(2H)-yl)ethan-1-one Hydrogen Bromide (MXL015)

¹H NMR (500 MHz, DMSO-d₆) δ 9.51 (br s, 2H), 8.20 (s, 1H), 8.01 (d, J=7.9 Hz, 1H), 7.95 (d, J=7.9 Hz, 1H), 7.58 (app. t, J=7.9 Hz, 1H), 5.75 (s, 2H), 2.53 (m, 2H), 2.33 (m, 2H), 1.71 (m, 4H).

¹³C NMR (126 MHz, DMSO-d₆) δ 190.3, 168.3, 137.4, 136.3, 135.1, 131.6, 128.8, 127.8, 122.6, 115.0, 52.7, 22.8, 22.7, 22.4, 21.3.

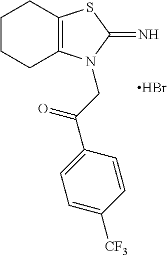 [SMILES: N=c1sc2c(n1CC(=O)c1ccc(C(F)(F)F)cc1)CCCC2 ; Br]

### 2-(2-Imino-4,5,6,7-tetrahydrobenzo[d]thiazol-3(2H)-yl)-1-(4-(trifluoromethyl)phenyl)ethan-1-one Hydrogen Bromide (MXL016)

¹H NMR (500 MHz, DMSO-d₆) δ 9.55 (br s, 2H), 8.23 (d, J=8.1 Hz, 2H), 8.01 (d, J=8.3 Hz, 2H), 5.80 (s, 2H), 2.53 (m, 2H), 2.34 (m, 2H), 1.71 (m, 4H).

¹³C NMR (126 MHz, DMSO-d₆) δ 190.6, 168.4, 135.1, 133.9 (q, J_{C-F}=32.1 Hz), 129.9, 128.8, 126.3, 124.2 (q, J_{C-F}=272.9 Hz), 115.0, 52.9, 22.8, 22.7, 22.4, 21.3.

### C. Synthesis of 2-(p-tolyl)-5,6,7,8-tetrahydroben-zo[d]imidazo[2,1-b]thiazole (Pifithrin β, MXL002)

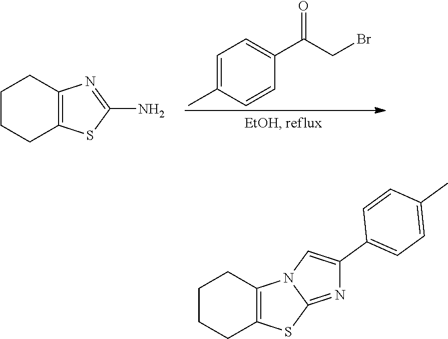

4,5,6,7-Tetrahydrobenzo[d]thiazol-2-amine (1 mmol, 154 mg) and 2-bromo-1-(p-tolyl)ethan-1-one (1 mmol, 213 mg) in dry EtOH (4 mL) was stirred at reflux for 90 min. A white precipitate formed when the reaction cooled. The precipitate was filtrated and washed with EtOH (2 mL×2), which afforded 209 mg (78% yield) of the desired product, 2-(p-tolyl)-5,6,7,8-tetrahydroben-zo[d]imidazo[2,1-b]thiazole (MXL002, Pifithrin j) as a white solid with.

¹H NMR (500 MHz, DMSO-d₆) δ 8.38 (s, 1H), 7.66 (m, 2H), 7.25 (m, 2H), 2.71 (m, 4H), 2.30 (s, 3H), 1.85 (m, 4H).

### D. Synthesis of N-(3-(2-oxo-2-(p-tolyl)ethyl)-4,5,6,7-tetrahydrobenzo[d]thiazol-2(3H)-ylidene)aceta-mide (Pifithrin α-Ac, MXL003)

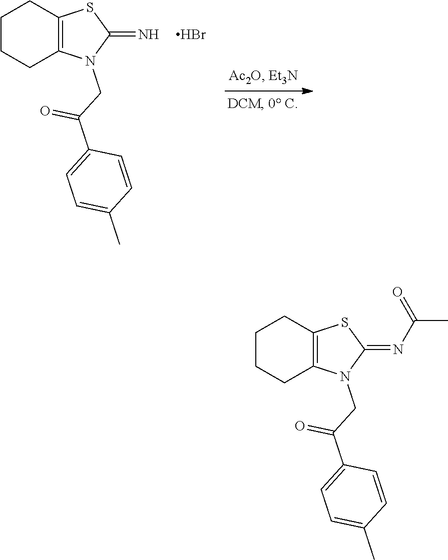 [SMILES: Cc1ccc(C(=O)Cn2c3c(sc2=N)CCCC3)cc1 ; Br ; CC(=O)/N=c1\sc2c(n1CC(=O)c1ccc(C)cc1)CCCC2]

To a solution of MXL001 (0.2 mmol, 72 mg) and Et₃N (0.4 mmol, 55.6 μL) in dichloromethane (5 mL) at 0° C. was added slowly acetic anhydride (0.4 mmol, 38 μL). The reaction was stirred for 1 h and TLC showed that no starting material was left. The reaction was quenched by adding 5 mL water and it was extracted with dichloromethane (5 mL×3). The combined organic phase was dried over Na₂SO₄ and concentrated using rotorvap. The residue was purified by column chromatography (2:1 hexanes:ethyl acetate), which generated 56 mg (85% yield) of the desired product, N-(3-(2-oxo-2-(p-tolyl)ethyl)-4,5,6,7-tetrahydrobenzo[d]thiazol-2(3H)-ylidene)aceta-mide (MXL003).

¹H NMR (300 MHz, CCCl₃) δ 7.99 (d, J=8.1 Hz, 2H), 7.38 (d, J=8.2 Hz, 2H), 5.56 (s, 2H), 2.61 (m, 2H), 2.50 (s, 3H), 2.39 (m, 2H), 2.20 (s, 3H), 1.89 (m, 4H).

### E. Synthesis of 3-(4-bromobenzyl)-4,5,6,7-tetrahydro-benzo[d]thiazol-2(3H)-imine Hydrogen Bromide (MXL005)

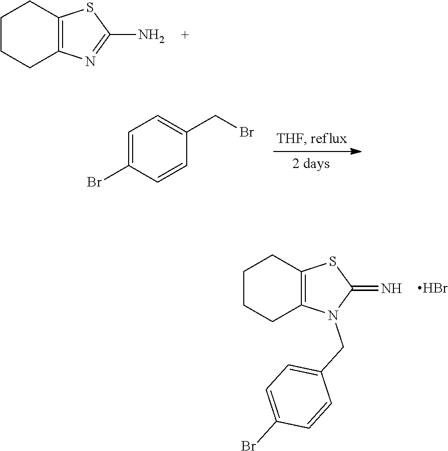 [SMILES: Nc1nc2c(s1)CCCC2 ; BrCc1ccc(Br)cc1 ; N=c1sc2c(n1Cc1ccc(Br)cc1)CCCC2 ; Br]

A mixture of 4,5,6,7-tetrahydrobenzo[d]thiazol-2-amine (0.33 mmol, 50.8 mg) and 4-bromo-benzyl bromide (0.33 mmol, 83.7 mg) in dry THF (2.5 mL) was stirred at reflux for 2 d. The white precipitate which formed during the reaction was filtrated and washed with THF (2 mL×2), which generated 99.5 mg (75% yield) of the desired product, 3-(4-bromobenzyl)-4,5,6,7-tetrahydro-benzo[d]thiazol-2(3H)-imine hydrogen bromide (MXL005) as a white solid.

¹H NMR (500 MHz, DMSO-d₆) δ 9.62 (br s, 2H), 7.58 (d, J=8.4 Hz, 2H), 7.08 (d, J=8.4 Hz, 2H), 5.23 (s, 2H), 2.46 (m, 2H), 2.30 (m, 2H), 1.66 (m, 4H).

¹³C NMR (126 MHz, DMSO-d₆) δ 167.7, 134.8, 133.8, 132.3, 128.9, 121.6, 115.7, 47.5, 22.8, 22.7, 22.1, 21.2.

### F. The Following Analogues (MXL006, MXL007 and MXL008) were Prepared Using a Similar Synthetic Route as for MXL005

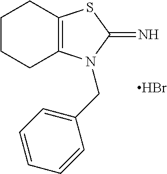 [SMILES: N=c1sc2c(n1Cc1ccccc1)CCCC2 ; Br]

### 3-Benzyl-4,5,6,7-tetrahydrobenzo[d]thiazol-2(3H)-imine Hydrogen Bromide (MXL006)

¹H NMR (500 MHz, DMSO-d₆) δ 9.60 (br s, 2H), 7.40 (app. t, J=7.4 Hz, 2H), 7.33 (app. t, J=7.4 Hz, 1H), 7.13 (d, J=7.3 Hz, 2H), 5.26 (s, 2H), 2.48 (m, 2H), 2.33 (m, 2H), 1.68 (m, 4H).

¹³C NMR (126 MHz, DMSO-d₆) δ 167.8, 135.1, 134.5, 129.6, 128.6, 126.7, 115.8, 48.2, 22.9, 22.8, 22.3, 21.3.

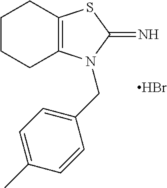 [SMILES: Cc1ccc(Cn2c3c(sc2=N)CCCC3)cc1 ; Br]

### 3-(4-Methylbenzyl)-4,5,6,7-tetrahydrobenzo[d]thiazol-2(3H)-imine Hydrogen Bromide (MXL007)

¹H NMR (500 MHz, DMSO-d₆) δ 9.59 (br s, 2H), 7.19 (d, J=7.8 Hz, 2H), 7.02 (d, J=7.8 Hz, 2H), 5.21 (s, 2H), 2.48 (m, 2H), 2.33 (m, 2H), 2.27 (s, 3H), 1.67 (m, 4H).

¹³C NMR (126 MHz, DMSO-d₆) δ 167.7, 137.9, 135.1, 134.1, 130.1, 126.8, 115.8, 48.0, 23.0, 22.8, 22.3, 21.4, 21.1.

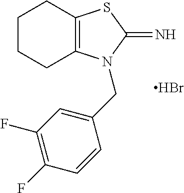 [SMILES: N=c1sc2c(n1Cc1ccc(F)c(F)c1)CCCC2 ; Br]

### 3-(3,4-Difluorobenzyl)-4,5,6,7-tetrahydrobenzo[d]thiazol-2(3H)-imine Hydrogen Bromide (MXL008)

¹H NMR (500 MHz, DMSO-d₆) δ 9.62 (br s, 2H), 7.47 (m, 1H), 7.32 (m, 1H), 6.97 (m, 1H), 5.24 (s, 2H), 2.48 (m, 2H), 2.33 (m, 2H), 1.69 (m, 4H).

¹³C NMR (126 MHz, DMSO-d₆) δ 168.0, 150.0 (dd, J_{C-F}=246.8, 15.4 Hz), 149.6 (dd, J_{C-F}=246.3, 12.5 Hz), 134.9, 132.2 (dd, J_{C-F}=5.7, 3.8 Hz), 123.7 (dd, J_{C-F}=6.7, 3.5 Hz), 118.7 (d, J_{C-F}=17.4 Hz), 116.6 (d, J_{C-F}=18.1 Hz), 115.9, 47.3, 22.96, 22.90, 22.3, 21.4.

hESC and hiPSC Culture and Organoid Generation

All hPSC experiments were conducted following prior approval from the University of California Los Angeles (UCLA) Embryonic Stem Cell Research Oversight Committee (ESCRO) and Institutional Review Board. Cortex (Cx) and ganglionic eminence (GE) organoids were generated from the H9 hPSC line²⁵ or Rett hiPSCs¹⁴ using methods in the art and outlined in schematic form in FIG. 1. Fusion was performed with minor modifications using methods in the art. Cx and GE organoids were cut at Day 56 and two halves (e.g., Cx+GE or Cx+Cx) were combined in a microcentrifuge tube containing 300 μl of N2B27 media⁶ and placed in a hyperoxic incubator containing 5% CO₂ and 40% 02 for 72 hours. Fused organoids were then carefully transferred to 24-well oxygen permeable dishes (Lumox, Sarstedt) and maintained in a hyperoxic environment with media changes every other day until their use. Neuron migration experiments were conducted by infection of either a Cx or GE organoid with 5 μL of about 1.98×10¹³ ml⁻¹ AAV1-TdTomato (pENN.AAV.CAG.tdTomato.WPRE.SV40, a gift of Dr. James M. Wilson, University of Pennsylvania Vector Core AV-1-PV3365) on Day 56 and fusion was performed as described 3 days after infection.

Prior to fusion, H9 hESC or non-mutant hiPSC derived Cx organoids express canonical cortical progenitor (PAX6, TBR2) and deep neuronal layer markers such as CTIP2 at Day 56. Expression of both deep (TBR1, BHLHB5) and superficial layer markers (SATB2) can be seen at Day 106. Unfused GE organoids express migratory interneuron progenitor markers (NKX2.1, DLX1, OLIG2) at Day 56, and both GE progenitor and mature interneuron markers (DLX1, DLX2, GAD65) at Day 98. Prior to fusion, D56 Cx or GE organoids were infected with AAV1-CAG:TdTomato virus, allowing for tracking of cells emanating from each compartment. Two weeks after fusion, labeled Cx cells showed limited migration into adjacent Cx or GE organoids. In comparison, labeled GE progenitors display robust migration and colonization of their Cx partner. Cx+Cx organoids at Day 106 express the superficial cortical marker SATB2, but not the migratory interneuron marker DLX5 or the differentiated interneuron maker GAD65. By contrast, the Cx end of Day 106 Cx+GE organoids show intermixing of SATB2, with DLX5⁺ GAD65⁺ cells. At Day 84, Cx+Cx organoids contain numerous excitatory synapses reflected by prominent colocalization of the pre- and post-synaptic markers VGLUT1 and PSD95, yet sparse numbers of inhibitory synapses detected by VGAT/GEPHYRIN co-staining. Cx+GE organoids on the other hand contain numerous VGLUT1⁺/PSD95⁺ excitatory and VGAT/GEPHYRIN⁺ inhibitory.

Cx+GE organoids demonstrate complex neural network activities. Addition of 100 μM of the GABAA antagonist bicuculline methiodide (BMI) had a minimal effect on Cx+Cx organoids, whereas BMI resulted in spontaneous synchronization of neural activities in Cx+GE organoids. Local field potentials (LFP) measured from a representative Cx+GE organoid revealed robust oscillatory activities at multiple frequencies during a 5-minute period, reflected in both raw traces and spectrogram. Spectral density analysis demonstrated the presence of multiple low frequency oscillations ranging from about 1-100 Hz. Cx+Cx organoids by contrast lack measurable oscillatory activities.

Rett syndrome fusion organoids have a higher density of excitatory synapses and exhibit hypersynchronous neural network activity. Isogenic Cx and GE organoids from Rett syndrome patient hiPSC that either contain (iCtrl) or lack (Mut) MECP2 expression were generated. iCtrl and Mut Cx organoids exhibited comparable formation of neural progenitors (SOX2, TBR2) and both deep and superficial layer neurons (CTIP2, BRN2). GE organoids displayed comparable expression of inhibitory interneuron markers (GAD65, SST, GABA). At about Day 100 unfused iCtrl and Mut Cx organoids show minimal expression of GAD65 expression. By contrast, about 20-25% of the cells in the Cx end of aged matched Cx+GE organoids express GAD65. Quantification of colocalized excitatory pre/post (VGLUT1/PSD) and inhibitory (VGAT/GEPHYRIN) puncta revealed a significant increase in colocalized excitatory puncta in Mut Cx+GE organoids. Representative raw (right) and post-processed colocalization images puncta used for quantification (left) are displayed. Panel f, Plots of the number of synapses per cell Data were pooled from multiple organoids. VGLUT1/PSD95, n=3 organoids for iCtrl and n=3 for Mut, 1180 cells) VGAT/GEPHYRIN, n=4 organoids for iCtrl and n=4 organoids for Mut, 1654 cells, *P=0.0244. Panel f, Mut Cx+GE organoids exhibit spontaneous synchronized Ca²⁺ transients that are not seen in iCtrl Cx+GE organoids. Panel g, Pooled data quantifications, n=7 iCtrl, n=10 Mut **P=0.0032 for synchronized transients, **P=0.0012 for multi-spiking neurons.

Generation of Rett hiPSCs

Rett iPSCs were derived from fibroblast line GM07982 obtained from Coriell Repositories and generated by lentiviral transduction of the cells with the Yamanaka factors (Oct4, Klf4, Sox2, and cMyc) using methods in the art. GM07982 cells were isolated from a 25-year-old female noted to have EEG abnormalities, and found to contain a truncating frameshift mutation, 705delG, in the MECP2 gene. As Rett females are typically heterozygous for the MECP2 mutation, the collected fibroblasts are mosaic in their MECP2 status with approximately half of the cells expressing the non-mutant allele. Unlike murine cells, the inactive X chromosome remains inactive after reprograming to pluripotency, allowing the generation MECP2 mutant (mut) and iCtrl (isogenic control) hiPSCs from the same patient fibroblasts.

### Immunohistochemistry

Organoids were immersion fixed in 4% paraformaldehyde, cryoprotected in 30% sucrose, frozen in Tissue-Tek Optimal Cutting Temperature (O.C.T., Sakura) media, and cryosectioned. Immunostaining was performed using methods in the art. Primary antibodies used include the following: goat anti-BRN2 (POU3F2; Santa Cruz Biotechnology sc-6029), 1:4000; mouse anti-CALBINDIN (Clone CB-955, Sigma-Aldrich C9848), 1:5000; rabbit anti-CALRETININ (EMD Millipore AB5054), 1:2000; mouse anti-CAM Kinase a (clone 6G9, Cell Signaling Technologies 50049S), 1:200; rat anti-CTIP2 (BCL11B; Abcam abl8465), 1:1000; mouse anti-CUX1 (CDP, clone B-10, Santa Cruz Biotechology sc-5140008), 1:100; rabbit anti-DLX1 (gift of Drs. Soo Kyung Lee and Jae Lee), 1:3000; guinea pig anti-DLX2 (gift of Drs. Kazuaki Yoshikawa and Hideo Shinagawa), 1:3000; guinea pig anti-DLX5 (gift of Drs. Kazuaki Yoshikawa and Hideo Shinagawa), 1:3000; rabbit anti-FOXG1 (Abcam abl8259), 1:1000; rabbit anti-GABA (Sigma-Aldrich A2052), 1:10000; mouse anti-GAD65 (BD Biosciences 559931), 1:200; mouse anti-GEPHYRIN (Synaptic Systems 147021), 1:500; goat anti-LHX2 (C-20, Santa Cruz Biotechnology sc-19344), 1:1000; mouse anti-LHX6 (Santa Cruz Biotechnology, sc271433), 1:200; N-CADHERIN (CDH2, BD Biosciences 610920), 1:1000; mouse anti-NKX2.1 (Novocastra NCL-L-TTR-1), 1:500; mouse anti-PAX6 (Developmental Studies Hybridoma Bank), 1:100; rabbit anti-PAX6 (MBL International PD022), 1:1000; mouse anti-PSD95 (Millipore MAB1598), 1:1000; mouse anti-SATB2 (Abcam ab51502), 1:100; goat anti-SOX2 (Santa Cruz Biotechnology sc-17320, 1:100; rat anti-SOMATOSTATIN (SST, EMD Millipore MAB354), 1:100; rabbit anti-TBR1 (Abcam ab31940), 1:2000; chicken anti-TBR2 (EOMES; EMD Millipore AB15894), 1:1000; guinea pig anti-VGAT (Synaptic Systems 131004), 1:1000; guinea pig anti-VGLUT1 (SLC17A7; EMD Millipore AB5905), 1:1000; VGLUT2 (SLC17A6; Synaptic Systems 135404), 1:8000. Secondary antibody staining was conducted using Dylight 405-, FITC, Alexa 488-, Cy3-, Alexa 594-, Cy5-, Alexa 647, Dylight 647-conjugated donkey anti-species-specific IgG or IgM antibodies (Jackson ImmunoResearch or Invitrogen/Molecular Probes). Nuclei were often counterstained using Hoechst 33258 added to the secondary antibody mix at a final concentration of 1 μg ml⁻¹. Images were primarily obtained on a Zeiss LSM 800 confocal microscope except for synaptic puncta, which were imaged using a 63× objective on a Zeiss LSM 880 confocal microscope equipped with Airyscan technology. All images that were directly compared were obtained with identical laser power settings. Image adjustments were limited to brightness, contrast, and level and were applied equally to all images in a set being compared.

Immunohistochemical analyses revealed similar cell composition in iCtrl and Mut fusion organoids. At about Day 100 iCtrl and Mut Cx+GE organoids have comparable numbers of GAD65⁺ positive cells in both the GE and Cx end. At about Day 100, both unfused Mut and unfused iCtrl GE organoids contain multiple interneuron subtypes including calretinin, calbindin, and somatostatin (SST) expressing cells. At about Day 100, Mut and iCtrl 100 GE and Cx organoids also contain GFAP⁺ astrocytes.

### Cell and Synaptic Puncta Quantification

All cell quantifications were obtained using at least 9 images per sample consisting of 3 non-contiguous regions per image and at least 3 images obtained from independent experiments. For GAD65 quantification, tiled images of fusion or unfused organoids were visualized in Photoshop (Adobe), a box of equal size was used to demarcate regions of interest on the outer edges of organoids, and total numbers of GAD65⁺ cells and HOECHST⁺ nuclei within the boxed region were manually tabulated. Synaptic puncta were identified and colocalized using Imaris image processing software (Bitplane) using the “spots” identifier, set to detect identically sized objects and thresholded against HOESCHT staining to exclude any nuclear overlap. The native “colocalization” function on Imaris was used to identify overlapping puncta.

### Live Organoid Calcium Imaging

The genetically encoded calcium indicator GCaMP6f was introduced into organoids between Days 88-95 via infection with 5 μl of 1.98×10¹³ GC ml⁻¹ pAAV1.Syn.GCaMP6f.WPRE.SV40 virus (gift from Dr. Douglas Kim & the GENIE Project (Addgene viral prep #100837-AAV1 or UPENN Vector core AV-1-PV2822). All imaging was performed 12-14 days after infection using a Scientifica two-photon microscope with a Coherent Chameleon tunable laser. Calcium transients were recorded at an excitation of 920 nm using a 20× water-immersion objective (Nikon) and a resonant scanner at 31 Hz with 512×512 pixel resolution and 0.5×0.5 mm field of view. Select organoids were subjected to the GABA type A receptor antagonist gabazine (25 μM) or bicuculline methiodide (100 μM).

### Microcircuit Identification

Raw Ca²⁺ imaging files in tiff format were processed to identify fluorescence transients (ΔF/F₀) and spike estimation in MATLAB (Mathworks Inc.) using the constrained non-negative matrix factorization-extended (CNMF-E) methodology known in the art. Following CNMF-E based data extraction neuronal microcircuit clusters were identified by performing hierarchical clustering on the correlation matrix of neuronal Ca²⁺ spiking data and analyzed using methods in the art.

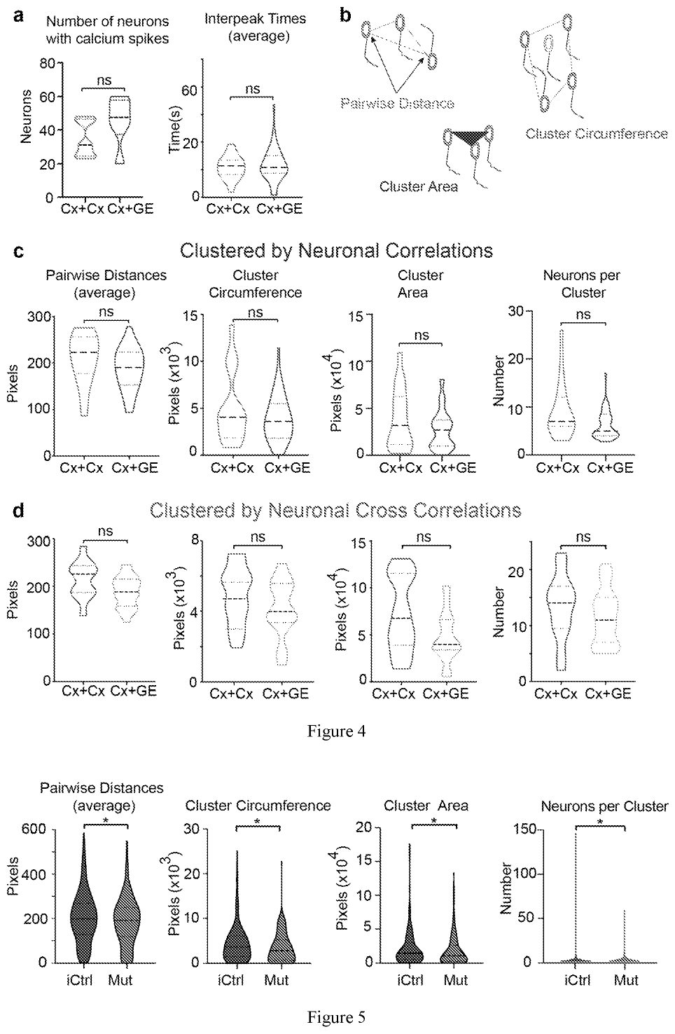

In brief, following generation of the correlation matrix, linkage analysis using the native MATLAB linkage function was to identify microcircuit clusters. Clusters were selected using an identical linkage parameter (1.5) in all experimental groups. Prior to deployment of this approach, both cross correlation and simple correlation were used to identify clusters from the same datasets and resulting clusters were visually inspected. Both approaches resulted in similar outcome measures (FIG. 4, Panels c,d), however cross correlation more consistently identified visually distinct clusters and was selected for final analyses. Synchronization of fluorescent transients was performed in MATLAB by tabulating the percentage of simultaneously active neurons in consecutive 4-frame long windows. A threshold of at least 40% simultaneous firing was used to delineate “synchronized” activity.

### Extracellular Recording

Organoids were recorded between about Days 100-107. Live organoids were perfused with 500 nM Kainate infusion in aCSF to initiate oscillatory network activity and activity was recorded using a patch pipette filled with aCSF connected with a headstage to a field amplifier (A-M Systems Inc., model 3000), and band pass filtered between 0.1 and 1000 Hz by to an instrumentation amplifier (Brownlee BP Precision, model 210A). Field potentials were digitized at 4096 Hz with a National Instruments A/D board using EVAN (custom-designed LabView-based software from Thotec) and analyzed with custom procedures (Wavemetrics, Igor Pro 8). Lower frequency activity was visualized for 10-minute epochs using power spectral densities (PSDs), which were calculated using the “dsperiodogram” function, and spectrograms, which were generated using the Gabor method on Igor Pro. High frequency activity up to 650 Hz was visualized by generating Morlet plots of 0.5-1.0 second epochs of the raw tracing used for low frequency analyses. Inter-spike intervals and spike frequencies were tabulated on Igor Pro using the identical 10-minute epochs used above.

### Statistical Information

All samples were subject to Shapiro-Wilk normality testing. Non-normal samples were analyzed by a two-tailed Mann-Whitney U-test, normally distributed data were analyzed by a two-tailed Student's t-test. Figure legends specify sample numbers and P values for all statistical tests. Each n represents an independent experiment.

### REFERENCES

The following references are herein incorporated by reference in their entirety with the exception that, should the scope and meaning of a term conflict with a definition explicitly set forth herein, the definition explicitly set forth herein controls:
1. Eiraku, M. & Sasai, Y. Self-formation of layered neural structures in three-dimensional culture of ES cells. Curr Opin Neurobiol 22, 768-777 (2012).
2. Di Lullo, E. & Kriegstein, A. R. The use of brain organoids to investigate neural development and disease. Nat Rev Neurosci 18, 573-584 (2017).
3. Bagley, J. A., Reumann, D., Bian, S., Levi-Strauss, J. & Knoblich, J. A. Fused cerebral organoids model interactions between brain regions. Nat Methods 14, 743-751 (2017).
4. Birey, F. et al. Assembly of functionally integrated human forebrain spheroids. Nature 545, 54-59 (2017).
5. Xiang, Y. et al. Fusion of regionally specified hPSC-derived organoids models human brain development and interneuron migration. Cell Stem Cell 21, 383-398 e387 (2017).
6. Watanabe, M. et al. Self-organized cerebral organoids with human-specific features predict effective drugs to combat Zika virus infection. Cell Rep 21, 517-532 (2017).
7. Pnevmatikakis, E. A. et al. Simultaneous denoising, deconvolution, and demixing of calcium imaging data. Neuron 89, 285-299 (2016).
8. Zhou, P. et al. Efficient and accurate extraction of in vivo calcium signals from microendoscopic video data. Elife 7 (2018).
9. Leonard, H., Cobb, S. & Downs, J. Clinical and biological progress over 50 years in Rett syndrome. Nat Rev Neurol 13, 37-51 (2017).
10. Mellios, N. et al. MeCP2-regulated miRNAs control early human neurogenesis through differential effects on ERK and AKT signaling. Mol Psychiatry 23, 1051-1065 (2018).
11. Armstrong, D. D., Dunn, K. & Antalffy, B. Decreased dendritic branching in frontal, motor and limbic cortex in Rett syndrome compared with trisomy 21. J Neuropathol Exp Neurol 57, 1013-1017 (1998).
12. Belichenko, P. V. et al. Widespread changes in dendritic and axonal morphology in Mecp2-mutant mouse models of Rett syndrome: evidence for disruption of neuronal networks. J Comp Neurol 514, 240-258 (2009).
13. Marchetto, M. C. et al. A model for neural development and treatment of Rett syndrome using human induced pluripotent stem cells. Cell 143, 527-539 (2010).
14. Ohashi, M. et al. Loss of MECP2 leads to activation of p53 and neuronal senescence. Stem Cell Reports 10, 1453-1463 (2018).
15. Sahara, S., Yanagawa, Y., O'Leary, D. D. & Stevens, C. F. The fraction of cortical GABAergic neurons is constant from near the start of cortical neurogenesis to adulthood. J Neurosci 32, 4755-4761 (2012).
16. Lu, H. et al. Loss and gain of MeCP2 cause similar hippocampal circuit dysfunction that is rescued by deep brain stimulation in a Rett syndrome mouse model. Neuron 91, 739-747 (2016).
17. Feldt Muldoon, S., Soltesz, I. & Cossart, R. Spatially clustered neuronal assemblies comprise the microstructure of synchrony in chronically epileptic networks. Proc Natl Acad Sci USA 110, 3567-3572 (2013).
18. Bragin, A., Engel, J., Jr., Wilson, C. L., Fried, I. & Buzsaki, G. High-frequency oscillations in human brain. Hippocampus 9, 137-142 (1999).
19. Bragin, A., Wilson, C. L., Almajano, J., Mody, I. & Engel, J., Jr. High-frequency oscillations after status epilepticus: epileptogenesis and seizure genesis. Epilepsia 45, 1017-1023 (2004).
20. Ito-Ishida, A., Ure, K., Chen, H., Swann, J. W. & Zoghbi, H. Y. Loss of MeCP2 in parvalbumin- and somatostatin-expressing neurons in mice leads to distinct Rett syndrome-like phenotypes. Neuron 88, 651-658 (2015).
21. Krajnc, N. Management of epilepsy in patients with Rett syndrome: perspectives and considerations. Ther Clin Risk Manag 11, 925-932 (2015).
22. Matsumoto, J. Y. et al. Network oscillations modulate interictal epileptiform spike rate during human memory. Brain 136, 2444-2456 (2013).
23. Verret, L. et al. Inhibitory interneuron deficit links altered network activity and cognitive dysfunction in Alzheimer model. Cell 149, 708-721 (2012).
24. Vignoli, A. et al. Effectiveness and tolerability of antiepileptic drugs in 104 girls with Rett syndrome. Epilepsy Behav 66, 27-33 (2017).
25. Thomson, J. A. et al. Embryonic stem cell lines derived from human blastocysts. Science 282, 1145-1147 (1998).
26. Tchieu, J. et al. Female human iPSCs retain an inactive X chromosome. Cell Stem Cell 7, 329-342 (2010).
27. Rousso, D. L., Gaber, Z. B., Wellik, D., Morrisey, E. E. & Novitch, B. G. Coordinated actions of the forkhead protein Foxp1 and Hox proteins in the columnar organization of spinal motor neurons. Neuron 59, 226-240 (2008).
28. Lee, B. et al. Dlx1/2 and Otp coordinate the production of hypothalamic GHRH- and AgRP-neurons. Nature Communications 9, 2026 (2018).
29. Kuwajima, T., Nishimura, I. & Yoshikawa, K. Necdin promotes GABAergic neuron differentiation in cooperation with Dlx homeodomain proteins. J Neurosci 26, 5383-5392 (2006).
30. Chen, T. W. et al. Ultrasensitive fluorescent proteins for imaging neuronal activity. Nature 499, 295-300 (2013).
31. Samarasinghe, R. A., et al. Identification of neural oscillations and epileptiform changes in human brain organoids. bioRxiv 820183 (2019).

All scientific and technical terms used in this application have meanings commonly used in the art unless otherwise specified.

Except when specifically indicated, peptides are indicated with the N-terminus on the left and the sequences are written from the N-terminus to the C-terminus. Similarly, except when specifically indicated, nucleic acid sequences are indicated with the 5′ end on the left and the sequences are written from 5′ to 3′.

As used herein, “Pifithrin compounds” include Pifithrin α and Pifithrin analogues as described herein. As used herein, a “Pifithrin analogue” refers to a compound having the following structural formula (A) as part of its structural backbone:

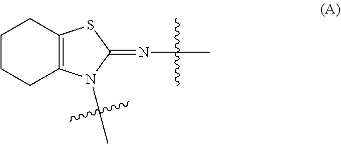 [SMILES: *N=c1sc2c(n1*)CCCC2]

Pifithrin analogues include compounds MXL002, MXL003, MXL004, MXL005, MXL006, MXL007, MXL008, MXL009, MXL010, MXL011, MXL012, MXL013, MXL014, MXL015, and MXL016 as described herein.

As used herein, the terms “subject”, “patient”, and “individual” are used interchangeably to refer to humans and non-human animals. The terms “non-human animal” and “animal” refer to all non-human vertebrates, e.g., non-human mammals and non-mammals, such as non-human primates, horses, sheep, dogs, cows, pigs, chickens, and other veterinary subjects and test animals. In some embodiments, the subject is a mammal. In some embodiments, the subject is a human.

The use of the singular can include the plural unless specifically stated otherwise. As used in the specification and the appended claims, the singular forms “a”, “an”, and “the” can include plural referents unless the context clearly dictates otherwise.

As used herein, “and/or” means “and” or “or”. For example, “A and/or B” means “A, B, or both A and B” and “A, B, C, and/or D” means “A, B, C, D, or a combination thereof” and said “A, B, C, D, or a combination thereof” means any subset of A, B, C, and D, for example, a single member subset (e.g., A or B or C or D), a two-member subset (e.g., A and B; A and C; etc.), or a three-member subset (e.g., A, B, and C; or A, B, and D; etc.), or all four members (e.g., A, B, C, and D).

As used herein, the phrase “one or more of”, e.g., “one or more of A, B, and/or C” means “one or more of A”, “one or more of B”, “one or more of C”, “one or more of A and one or more of B”, “one or more of B and one or more of C”, “one or more of A and one or more of C” and “one or more of A, one or more of B, and one or more of C”.

As used herein, the phrase “consists essentially of” in the context of neural cells having a loss of function mutation in the Methyl-CpG Binding Protein 2 (MECP2) gene means that the neural cells may have other genetic mutations so long as the mutations do not affect the phenotype of the MECP2⁻ mutation. In the context of a given ingredient in a composition, “consists essentially of” means that the composition may include additional ingredients so long as the additional ingredients do not adversely impact the activity, e.g., biological or pharmaceutical function, of the given ingredient.

The phrase “comprises, consists essentially of, or consists of A” is used as a tool to avoid excess page and translation fees and means that in some embodiments the given thing at issue: comprises A, consists essentially of A, or consists of A. For example, the sentence “In some embodiments, the composition comprises, consists essentially of, or consists of A” is to be interpreted as if written as the following three separate sentences: “In some embodiments, the composition comprises A. In some embodiments, the composition consists essentially of A. In some embodiments, the composition consists of A.”

Similarly, a sentence reciting a string of alternates is to be interpreted as if a string of sentences were provided such that each given alternate was provided in a sentence by itself. For example, the sentence “In some embodiments, the composition comprises A, B, or C” is to be interpreted as if written as the following three separate sentences: “In some embodiments, the composition comprises A. In some embodiments, the composition comprises B. In some embodiments, the composition comprises C.” As another example, the sentence “In some embodiments, the composition comprises at least A, B, or C” is to be interpreted as if written as the following three separate sentences: “In some embodiments, the composition comprises at least A. In some embodiments, the composition comprises at least B. In some embodiments, the composition comprises at least C.”

To the extent necessary to understand or complete the disclosure of the present invention, all publications, patents, and patent applications mentioned herein are expressly incorporated by reference therein to the same extent as though each were individually so incorporated.

Having thus described exemplary embodiments of the present invention, it should be noted by those skilled in the art that the within disclosures are exemplary only and that various other alternatives, adaptations, and modifications may be made within the scope of the present invention. Accordingly, the present invention is not limited to the specific embodiments as illustrated herein, but is only limited by the following claims.

1. A compound having Formula I or Formula II 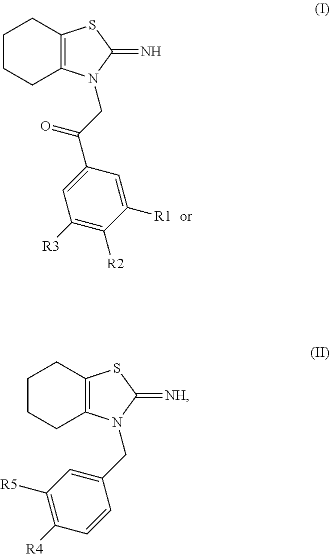
R2 (I) or R4 (II), wherein
(a) R1 is Cl or Br and R2 and R3 are H;
(b) R2 is a trimethylsilyl group (TMS), and R1 and R3 are H;
(c) R1 and R3 are CF3 and R2 is H;
(d) R4 is Br and R5 is H; or
(e) R4 and R5 are F; and
pharmaceutically acceptable salts, solvates, and prodrugs thereof.

2. A composition comprising one or more compounds according to claim 1, and a pharmaceutically acceptable carrier.

3. A method of treating Rett Syndrome in a subject, which comprises administering to the subject at least one Pifithrin compound or a composition thereof.

4. The method according to claim 3, wherein the Pifithrin compound is
2-(2-imino-4,5,6,7-tetrahydrobenzo[d]thiazol-3(2H)-yl)-1-(p-tolyl)ethan-1-one hydrogen bromide (Pifithrin α);
2-(p-tolyl)-5,6,7,8-tetrahydroben-zo[d]imidazo[2,1-b]thiazole (Pifithrin β);
N-(3-(2-oxo-2-(p-tolyl)ethyl)-4,5,6,7-tetrahydrobenzo[d]thiazol-2(3H)-ylidene)aceta-mide (Pifithrin α-Ac);
2-(2-Imino-4,5,6,7-tetrahydrobenzo[d]thiazol-3(2H)-yl)-1-(4-(trimethylsilyl)phenyl)ethan-1-one hydrogen bromide;
3-(4-bromobenzyl)-4,5,6,7-tetrahydro-benzo[d]thiazol-2(3H)-imine hydrogen bromide;
3-Benzyl-4,5,6,7-tetrahydrobenzo[d]thiazol-2(3H)-imine hydrogen bromide;
3-(4-Methylbenzyl)-4,5,6,7-tetrahydrobenzo[d]thiazol-2(3H)-imine hydrogen bromide;
3-(3,4-Difluorobenzyl)-4,5,6,7-tetrahydrobenzo[d]thiazol-2(3H)-imine hydrogen bromide;
1-(3-Fluorophenyl)-2-(2-imino-4,5,6,7-tetrahydrobenzo[d]thiazol-3(2H)-yl)ethan-1-one hydrogen bromide;
2-(2-Imino-4,5,6,7-tetrahydrobenzo[d]thiazol-3(2H)-yl)-1-(3-nitrophenyl)ethan-1-one hydrogen bromide;
2-(2-Imino-4,5,6,7-tetrahydrobenzo[d]thiazol-3(2H)-yl)-1-(4-methoxyphenyl)ethan-1-one hydrogen bromide;
1-(3-Chlorophenyl)-2-(2-imino-4,5,6,7-tetrahydrobenzo[d]thiazol-3(2H)-yl)ethan-1-one hydrogen bromide;
1-(3,4-Dichlorophenyl)-2-(2-imino-4,5,6,7-tetrahydrobenzo[d]thiazol-3(2H)-yl)ethan-1-one hydrogen bromide;
1-(3,5-Bis(trifluoromethyl)phenyl)-2-(2-imino-4,5,6,7-tetrahydrobenzo[d]thiazol-3(2H)-yl)ethan-1-onehydrogen bromide;
1-(3-Bromophenyl)-2-(2-imino-4,5,6,7-tetrahydrobenzo[d]thiazol-3(2H)-yl)ethan-1-one hydrogen bromide; or
2-(2-Imino-4,5,6,7-tetrahydrobenzo[d]thiazol-3(2H)-yl)-1-(4-(trifluoromethyl)phenyl)ethan-1-one hydrogen bromide.

5. The method according to claim 3, wherein the Pifithrin compound is
2-(2-imino-4,5,6,7-tetrahydrobenzo[d]thiazol-3(2H)-yl)-1-(p-tolyl)ethan-1-one hydrogen bromide (Pifithrin a);
2-(p-tolyl)-5,6,7,8-tetrahydroben-zo[d]imidazo[2,1-b]thiazole (Pifithrin β);
N-(3-(2-oxo-2-(p-tolyl)ethyl)-4,5,6,7-tetrahydrobenzo[d]thiazol-2(3H)-ylidene)aceta-mide (Pifithrin α-Ac); or
2-(2-Imino-4,5,6,7-tetrahydrobenzo[d]thiazol-3(2H)-yl)-1-(4-(trimethylsilyl)phenyl)ethan-1-one hydrogen bromide.

6. The method according to claim 3, wherein the Pifithrin compound is
2-(2-imino-4,5,6,7-tetrahydrobenzo[d]thiazol-3(2H)-yl)-1-(p-tolyl)ethan-1-one hydrogen bromide (Pifithrin α);
3-(4-bromobenzyl)-4,5,6,7-tetrahydro-benzo[d]thiazol-2(3H)-imine hydrogen bromide;
3-Benzyl-4,5,6,7-tetrahydrobenzo[d]thiazol-2(3H)-imine hydrogen bromide;
3-(4-Methylbenzyl)-4,5,6,7-tetrahydrobenzo[d]thiazol-2(3H)-imine hydrogen bromide;
3-(3,4-Difluorobenzyl)-4,5,6,7-tetrahydrobenzo[d]thiazol-2(3H)-imine hydrogen bromide; or
1-(3-Fluorophenyl)-2-(2-imino-4,5,6,7-tetrahydrobenzo[d]thiazol-3(2H)-yl)ethan-1-one hydrogen bromide.

7. A brain fusion organoid comprising a cerebral cortex (Cx) organoid fused to a ganglionic eminence (GE) organoid.

8. The brain fusion organoid of claim 7, wherein the cerebral cortex (Cx) organoid and/or the ganglionic eminence (GE) organoid comprises, consists essentially of, or consists of neural cells having a loss of function mutation in the Methyl-CpG Binding Protein 2 (MECP2) gene.

9. An assay method for determining whether a given compound treats, inhibits, or reduces abnormal neural activity resulting from neural cells having a loss of function mutation in the Methyl-CpG Binding Protein 2 (MECP2) gene, which comprises contacting the brain fusion organoid of claim 8 with the given compound and comparing the resulting oscillatory activity with that of untreated controls.

10. The method of claim 2, wherein the Pifithrin compound has the following structural formula (A) 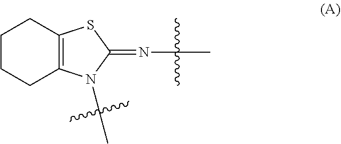 [SMILES: *N=c1sc2c(n1*)CCCC2]
as part of its structural backbone.
